(19) United States
(12) Patent Application Publication
(10) Pub. No.: US 2011/0052667 A1
(43) Pub. Date: Mar. 3, 2011

(54) COMPOSITIONS AND METHODS FOR INHIBITING ANGIOGENESIS AND TUMORIGENESIS

(75) Inventor: Pierluigi Scalia, Palmdale, CA (US)

(73) Assignee: Sylgen Laboratories, Inc., Palmdale, CA (US)

(21) Appl. No.: 12/666,624

(22) PCT Filed: Jun. 27, 2008
(86) PCT No.: PCT/US08/68646

§ 371 Date: Oct. 5, 2010

Related U.S. Application Data
(60) Provisional application No. 60/946,899, filed on Jun. 28, 2007.

(51) Int. Cl. A61K 9/127; C12Q 1/68; G01N 33/574; C12M 1/34; C12N 5/09; A61K 39/395; A61K 31/7105; A61P 35/00

(52) U.S. Cl. 424/450; 435/6; 435/7.23; 435/287.2; 435/325; 424/158.1; 514/44 A

(57) ABSTRACT

The present invention relates to the regulation of angiogenesis and tumorigenesis. The invention further relates to methods for identifying and using agents, such as small organic molecules, antibodies, peptides and nucleic acids (e.g., antisense molecules, RNAi molecules and ribozymes), that modulate angiogenesis and tumorigenesis via modulation of IGF-II and, in turn, EphB4, as well as to the use of expression profiles and compositions in the diagnosis and therapy of angiogenesis and cancer.

### CROSS-REFERENCES TO RELATED APPLICATIONS

This patent application claims the benefit of U.S. Provisional Patent Application No. 60/946,899, filed Jun. 27, 2008, the teachings of which are incorporated herein by reference for all purposes.

### STATEMENT AS TO RIGHTS TO INVENTIONS MADE UNDER FEDERALLY SPONSORED RESEARCH OR DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

The Insulin-like growth factor-II (IGF-II) is expressed in up to 80% of solid cancers (Rogler et al., Journal of Biological Chemistry, 269(19):13779-13784 (1994)). It has been directly involved in oncogene-induced tumor progression and malignant transformation (Christofori et al., Nature, 369:414-417, (1994)). Cancer-secreted IGF-II has been described in the majority of tumor patients displaying a paraneoplastic syndrome known as “non-islet cell tumor-induced hypoglycemia” or NICTH (Schweichler et al., Obstet. Gynecol., 85(5):810-813 (1995); and Van Doom et al., Clin. Chem., 48(10):1739-1750 (2002)). IGF-II expression in cancer has been correlated with higher grades or more malignant stages (Toretsky et al., Journal of Endocrinology, 149: 367-372 (1996); Bates et al., British Journal of Cancer, 72:1189-1193 (1995)), and its overexpression in mammals is responsible for a tissue overgrowth syndrome leading to an increased and rapid onset of solid tumors (Morison et al., Molecular Medicine Today, 4(3):110-115 (1998); Eggenshwiler et al., Genes Dev., 11:3128-3142 (1997)). However, the mechanism by which it induces tumor progression has not been fully established. The biologic effects of IGF-II were initially linked to the exclusive binding and activation of the IGF-IR (Steller et al., Cancer Res., 56(8):1761-1765, (1996); and Baserga, Cancer Res., 55:249-52 (1995)). It has now been shown, however, that IGF-II also binds with high affinity and activates an insulin receptor isoform variant (InsR-A), and that this mechanism is active in fetal and cancer cells and tissues (Frasca et al., Mol. Cell Biol., 19(5):3278-88 (1999)). This mechanism is likely to confer IGF-II with a wider spectrum of biological effects and growth advantages since it can alternatively and/or contemporaneously bind also the IGFI-R under the same conditions. However, in a number of cancers where the IGF-IR is expressed at low levels or is not expressed at significant levels, the role of the IGF-II/InsR-A activation loop has been shown to be essential in mediating IGF-II cellular effects (Sciacca et al., Oncogene, 18:2471-79 (1999); and Sciacca et al., Oncogene, 21:8240-50 (2002).

IGF-II has previously been described to possess intrinsic angiogenic activity (Lee et al, Br. J. Cancer, 82(2):385-91 (2000)). Several investigators have further established the role of the IGF-I receptor in mediating a signal leading to the upregulation of the Vascular Endothelial Growth Factor (VEGF). This activation loop explains, in part, the IGF-II angiogenic potential. However, it does not explain the lack of correlation between IGF-II and VEGF at the transitional “switch” between benign to malignant proliferation, suggesting the existence of additional mechanisms by which IGF-II exerts its blood vessel formation effect in cancers.

EphrinB2 and EphB4 are transmembrane proteins exclusively expressed in arterial (EphrinB2) or venous (EphB4) endothelial cells in normal adult vasculature (see, U.S. Pat. Nos. 6,864,227, 6,887,674, 6,916,625 and 6,579,683, all of which issued to Wang et al. and are entitled “Artery- and vein-specific proteins and uses thereof;” U.S. Pat. No. 6,555,321, which issued to Thomas et al. and is entitled “Methods for determining cell responses through EphB4 receptors”; and Hu et al., Cell, 93(5):741-753 (1998)). Their interaction is involved in the early formation of blood vessels (primitive vasculature) as well as in the maturation and phenotypic differentiation of arteries and veins.

In addition, it has now been demonstrated that these two molecules are ectopically expressed in solid cancers and their role in promoting tumor growth has been established (Masood et al., Blood, 105(3):1310-1318 (2005); and Sinha et al., Ear Nose Throat J., 82(11):866-887 (2003)).

The mechanism by which EphrinB2 and EphB4 exerts their biologic effect in tumors has been partially described (Noren et al., PNAS, 101(15):5583-5588 (2004). In brief, the ectodomain of EphB4 has been shown to attract endothelial cells in vitro and to induce angiogenesis through the direct binding and activation of EphrinB2. The recent description of circulating endothelial cells at different level of maturation in cancer (Rafii et al., Nature Reviews, 2:826-835 (2002); Bertolini et al., Nature Reviews, 6:835-45 (2006)) might justify a scenario wherein the EphB4/EphrinB2 system is recruiting CECs at the tumor site and is promoting blood vessel formation, thereby allowing the unrestricted tri-dimensional growth of the tumor.

Thus, there is a need in the art to understand more fully the mechanisms by which IGF-II and EphB4 exert their biological effects in angiogenesis and tumorigenesis, and to use this understanding of the mechanisms involved to develop compositions and methods that can be used to inhibit angiogenesis and/or tumorigenesis. Quite surprisingly, the present invention fulfills these needs as well as other needs.

### BRIEF SUMMARY OF THE INVENTION

It has now been discovered that cancer-secreted IGF-II is responsible for an autocrine- or paracrine-stimulatory loop (via expression of IGF-II tyrosine kinase receptors (i.e., IGF-II TK receptors, such as IGF-IR and/or InsR-A) in the same cell or in a cell that is in close cellular proximity to the cell, such as a tumor cell), resulting in the up-regulation of the EphB4 protein. More particularly, it has now been discovered that EphB4 is under the direct regulation and control of IGF-II in cancer and, thus, that IGF-II is a valuable therapeutic target in all those pathologic conditions displaying abnormal activity of EphB4. Importantly, the present invention provides evidence that IGF-II is not an alternative target to the IGF-IR targeting, but is, in fact, a preferred target in view of its ability to act through the InsR-A and its role in the autocrine- and paracrine-stimulatory loop that results in the up-regulation of EphB4.

More particularly, IGF-IR and InsR-A are found to be expressed in both cancer cells and in normal cells even though their expression level is generally increased in cancer cells. While IGF-II expression is initially restricted to the stromal or parenchymal tissue (e.g., fibroblasts) surrounding the tumor, all cancers at any stage (pre-cancerous to overt cancer) as well as normal tissues express one or both of IGF-IR and InsR-A, which also serve as receptors for IGF-I and insulin, respectively. This supports the finding that the IGF-IR and InsR-A receptors are not cancer-specific targets, but instead serve other physiological functions and roles. In contrast, the restricted expression of IGF-II at local tissue levels in cancer supports the findings of the present invention that IGF-II constitutes a preferred target to inhibit or block the IGF cancer-promoting and angiogenic effects in cancer. Clearly, based on the findings of the present invention, IGF-II is a preferred target over the block of one of its tyrosine kinase receptors (e.g., IGF-IR or InsR-A), which also serve to mediate the effects of their physiologic ligands (i.e., IGF-I and insulin).

The present invention provides for the first time the identification of a functional relationship or link between IGF-II, a IGF-II TK receptor (e.g., IGF-IR and/or InsR-A) and EphB4 in tumor growth at the benign, transitional and malignant stages, and the application of this function link for the detection of tumors, for tumor staging, for drug delivery and for the control of angiogenesis and tumorigenesis. In particular, the present invention provides methods for inhibiting cancer angiogenesis and/or proliferation by controlling EphB4 gene or protein expression through the inhibition of IGF-II (by blocking or inhibiting IGF-II gene transcript (RNA) or expressed or secreted protein). Using the methods and compositions of the present invention the angiogenic/proliferative function(s) of EphB4 in cancer cells can be controlled through the inhibition and/or blocking of IGF-II gene transcript (RNA) or expressed/secreted protein.

Thus, in one aspect, the present invention provides a method for qualifying the tumor status in a subject, the method comprising: (a) detecting the presence or absence of IGF-II, a IGF-II TK receptor (e.g., IGF-IR and/or InsR-A) and EphB4 in a biological sample from the subject; and (b) correlating the presence or absence of IGF-II, a IGF-II TK receptor (e.g., IGF-IR and/or InsR-A) and EphB4 with tumor status. In one embodiment, the tumor status is benign and the biological sample is IGF-II negative/IGF-II TK receptor (e.g., IGF-IR and/or InsR-A) positive/EphB4 negative. In another embodiment, the tumor status is transitional and the biological sample is IGF-II negative/IGF-II TK receptor (e.g., IGF-IR and/or InsR-A) positive/EphB4 positive. In this embodiment, the method further comprises detecting the presence or absence of IGF-II in parenchymal and/or stromal tissue (e.g., fibroblasts) surrounding the tumor, wherein the presence of IGF-II in surrounding parenchymal or stromal tissue further indicates that the tumor status is transitional. In yet another embodiment, the tumor status is malignant and the biological sample is IGF-II positive/IGF-II TK receptor (e.g., IGF-IR and/or InsR-A) positive/EphB4 positive.

In certain embodiments of the above method, IGF-II, the IGF-II TK receptor (e.g., IGF-IR and/or InsR-A) and EphB4 are detected by detecting RNA. In other embodiments, IGF-II, the IGF-II TK receptor (e.g., IGF-IR and/or InsR-A) and EphB4 are detected by detecting expressed protein. In connection with this later embodiment, IGF-II, the IGF-II TK receptor (e.g., IGF-IR and/or InsR-A) and EphB4 can be detected by immunoassay.

In one embodiment of the above method, the biological sample is a tumor cell or a biopsy of a solid tumor. In preferred embodiments, the tumor cell or solid tumor is from, for example, a carcinoma of the breast, kidney, thyroid, prostrate, liver, bone, lymph node, cervix, skin and colon or a sarcoma of the bone, muscle and lymphatic tissue.

In certain embodiments, the method further comprises (c) managing subject treatment based on the tumor status. In other embodiments, the method further comprises (c) reporting the tumor status to the subject. In still other embodiments, the method further comprises (c) recording the tumor status on a tangible medium. In yet other embodiments, the foregoing methods further comprise (d) detecting IGF-II, a IGF-II TK receptor (e.g., IGF-IR and/or InsR-A) and EphB4 after subject management and correlating the measurement with disease progression.

In another aspect, the present invention provides methods for identifying a compound that modulates angiogenesis in a cell that co-expresses IGF-II and EphB4 or in a cell wherein EphB4 expression is driven by IGF-II expression in the surrounding stromal or parenchymal tissue (e.g., fibroblasts), the method comprising the steps of (i) contacting the cell with a compound that inhibits IGF-II; and (ii) determining the functional effect of the compound upon EphB4 in the cell. In one embodiment, the compound inhibits IGF-II gene expression. In another embodiment, the compound inhibits IGF-II protein expression. In certain preferred embodiments, the compound includes, but is not limited to, an antibody, an RNAi molecule, an antisense molecule; and a small organic molecule.

Typically, in connection with the above methods, the compounds are screened for their ability to inhibit angiogenesis. In one embodiment, the functional effect is EphB4 gene expression. In another embodiment, the functional effect is EphB4 protein expression. It will be apparent to those of skill in the art that the function effect can include the identification of other markers of angiogenesis as described herein.

In another aspect, the present invention provides methods for identifying a compound that inhibits tumorigenesis in a tumor cell that co-expresses IGF-II and EphB4 or in a tumor cell wherein EphB4 expression is driven by IGF-II expression in the surrounding stromal or parenchymal tissue (e.g., fibroblasts), the method comprising the steps of: (i) contacting the tumor cell with a compound that inhibits IGF-II; and (ii) determining the functional effect of the compound upon EphB4 in the tumor cell. In one embodiment, the compound inhibits IGF-II gene expression. In another embodiment, the compound inhibits IGF-II protein expression. In certain preferred embodiments, the compound includes, but is not limited to, an antibody, an RNAi molecule, an antisense molecule; and a small organic molecule.

Typically, in connection with the above methods, the compounds are screened for their ability to inhibit tumorigenesis. In one embodiment, the functional effect is EphB4 gene expression. In another embodiment, the functional effect is EphB4 protein expression. It will be apparent to those of skill in the art that the function effect can include the identification of other markers of tumorigenesis as described herein.

In yet another aspect, the present invention provides kits for qualifying tumor status in a subject. In one embodiment, the kit comprising: (a) a first solid support comprising at least one capture reagent attached thereto, wherein the capture reagent binds IGF-II; (b) a second solid support comprising at least one capture reagent attached thereto, wherein the capture reagent binds a IGF-II TK receptor (e.g., IGF-IR and/or InsR-A); (c) a third solid support comprising at least one capture reagent attached thereto, wherein the capture reagent binds EphB4; and (d) instructions for using the first, second and third solid supports to detect IGF-II, a IGF-II TK receptor and EphB4.

In one embodiment, the capture reagent that binds IGF-II is an antibody. In one embodiment, the capture reagent that binds the IGF-II TK receptor is an antibody. In another embodiment, the capture reagent that binds EphB4 is an antibody. In another embodiment, the capture reagent that binds EphB4 is EphrinB2. In certain embodiment, the kits of the present invention further comprise (d) a container containing IGF-II, a IGF-II TK receptor or EphB4 or, ideally, three containers, the first of which contains IGF-II, the second of which contains a IGF-II TK receptor and the third of which contains EphB4.

In still another aspect, the present invention provides methods for inhibiting EphB4 gene or protein expression in a cell that co-expresses EphB4 and IGF-II or in a cell wherein EphB4 expression is driven by IGF-II expression in the surrounding stromal or parenchymal tissue (e.g., fibroblasts), the method comprising contacting the cell with a compound that inhibits IGF-II, thereby inhibiting EphB4 gene or protein expression. In one embodiment, the compound inhibits IGF-II gene expression. In another embodiment, the compound inhibits IGF-II protein expression. In certain preferred embodiments, the compound includes, but is not limited to, an antibody, an RNAi molecule, an antisense molecule; and a small organic molecule. In one embodiment, the cell is a tumor cell.

In another aspect, the present invention provides methods for inhibiting the growth of a tumor cell that co-expresses EphB4 and IGF-II or of a tumor cell wherein EphB4 expression is driven by IGF-II expression in the surrounding stromal or parenchymal tissue (e.g., fibroblasts), the method comprising contacting the tumor cell with a compound that inhibits IGF-II, thereby inhibiting the growth of the tumor cell. In one embodiment, the compound inhibits IGF-II gene expression. In another embodiment, the compound inhibits IGF-II protein expression. In certain preferred embodiments, the compound includes, but is not limited to, an antibody, an RNAi molecule, an antisense molecule; and a small organic molecule. In other preferred embodiments, the tumor cell is in a human.

In yet another embodiment, the present invention provides an EphB4-specific targeting composition, the composition comprising an EphB4 binding moiety and an IGF-II inhibitor (or blocking agent), wherein the EphB4 binding moiety is linked to the IGF-II inhibitor. In one embodiment, the EphB4 binding moiety is an antibody or EphrinB2. In another embodiment, the IGF-II inhibitor is a member selected from the group consisting of an antibody, a peptide, an antisense molecule, a RNAi molecule and a small organic molecule. In a preferred embodiment, the EphB4 binding moiety is EphrinB2 and the IGF-II inhibitor is an antibody.

In still another aspect, the present invention provides a method of inhibiting tumorigenesis in a cell that co-expresses IGF-II and EphB4 or in a cell wherein EphB4 expression is driven by IGF-II expression in the surrounding parenchymal or stromal tissue (e.g., fibroblasts), the method comprising contacting the cell with an EphB4-specific targeting compound, the compound comprising: (i) an EphB4 binding moiety; and (ii) an IGF-II inhibitor, wherein the EphB4 is linked to the IGF-II inhibitor.

In a further aspect, the present invention provides an EphB4-specific targeting vehicle, the vehicle comprising: a liposome; a EphB4 binding moiety, wherein the EphB4 binding moiety is on the outer surface of the liposome; and an IGF-II inhibitor (or blocking agent). In one embodiment, the EphB4 binding moiety is an antibody or EphrinB2. In another embodiment, the IGF-II inhibitor is a member selected from the group consisting of an antibody, a peptide, an antisense molecule, a RNAi molecule and a small organic molecule. In a preferred embodiment, the EphB4 binding moiety is EphrinB2 and the IGF-II inhibitor is an antibody.

In still another aspect, the present invention provides a method for inhibiting tumorigenesis in a cell that co-expresses IGF-II and EphB4 or in a tumor cell wherein EphB4 expression is driven by IGF-II expression in the surrounding parenchymal or stromal tissue (e.g., fibroblasts), the method comprising contacting the cell with an EphB4-specific targeting vehicle, the vehicle comprising: (i) a liposome; (ii) a EphB4 binding moiety, wherein the EphB4 binding moiety is on the outer surface of the liposome; and (iii) an IGF-II inhibitor or blocking agent.

In a further embodiment, the present invention provides a method for inhibiting the progression of a tumor cell, such as a benign tumor cell or a transitional tumor cell, towards a malignant state, wherein EphB4 expression is driven by IGF-II expression in surrounding parenchymal or stromal tissue, the method comprises contacting the surrounding parenchymal or stromal tissue with a compound that inhibits IGF-II, thereby inhibiting the progression of the tumor cell towards a malignant state.

In a further aspect, the present invention provides method for inhibiting the IGF-II mediated activation of EphB4 and its biologic effects in tumorigenesis (such as cancer) and angiogenesis via interference with, for example, the Homeobox (Hox) transcription factor HoxA9 and/or its cancer expressed orthologs and/or paralogs acting on the motif TAAT at position −1365 of the human EphB4 promoter. This includes both Hox and non-Hox bona fide transcription factors expressed in cancer and contextually acting through the TAAT motif at position −1365 of the human EphB4 promoter.

Other features, objects and advantages of the invention and its preferred embodiments will become apparent from a reading of the detailed description, examples, claims and figures that follow.

### BRIEF DESCRIPTION OF THE DRAWINGS

FIG. 1 demonstrates that block of secreted IGF-II by a neutralizing antibody strongly inhibits EphB4 protein expression in different Mesothelioma cell lines (52H=NCI−2052H; 211H=MSTO−211H; 373H=NCI−2373H, all of which are human Mesothelioma cell lines expressing both the IGF-IR and the InsR isoform A). FIG. 1A. EphB4 protein levels were measured by western blot as previously described (see, Scalia et al., infra (2001)). Briefly, Mesothelioma cell cultures previously cultured in 10% Fetal Bovine Serum were cultured either in the presence or absence of an anti-IGF2 neutralizing antibody (R&D) at 20 μg/ml concentration. After 24 hrs, the medium was removed and the cells harvested for total cell lysates preparation (TCL). The same amount of proteins from TCLs were then resolved on a 10% SDS-Polyacrilamide gel and transferred onto a PVDF membrane. EphB4 protein was detected using a rabbit anti-EphB4 antibody (Santacruz Biotechnology) and a horseradish-conjugated anti-rabbit secondary antibody. FIG. 1B. The same cell lines were also tested for total content of EphB4 along with other cell lines not expressing IGF-II. FIG. 1C. Co-expression of IGF-II and IGF-II tyrosine kinase receptors in mesothelioma cell lines by RT-PCR. 52H=NCI−2052H, human Mesothelioma cell line; 211H=MSTO−211H, human Mesothelioma cell line; and 373H=NCI−2373H, human Mesothelioma cell line. FIG. 1D. IGF-II protein expressed by Mesothelioma cell lines is secreted in the extracellular compartment in its high molecular weight variants.

FIG. 2 demonstrates that the venous marker EphB4 is regulated by IGF-II at the transcriptional gene level and this effect is mediated by the TAAT motif at position −1365 of the human EphB4 promoter within a specific binding site for the HoxA9 transcription factor and/or its cancer expressed mammalian orthologs and/or paralogs. The TAAT motif at position −1365 of the −1500/−7mut construct used in conditions 7-9 was substituted with a TGCT motif by site directed mutagenesis. In FIG. 2, Series 1: Firefly Luciferare signal (pGL3-Luc EphB4 constructs); Seies 2: Renilla Luciferase signal (mock vector); and RLU: Relative Luciferase Units. Conditions: 1-3: −7/−1508 EphB4 promoter region; 4-6: −7/−1028 EphB4 promoter region; 7-9: −7/−1508mut EphB4 promoter construct, wherein: 1. Serum Free, EphB4 promoter −7/−1508; 2. IGF-II, 10 nM, EphB4 promoter −7/−1508; 3.10% FBS, EphB4 promoter −7/−1508; 4. Serum Free, EphB4 promoter −7/−1028; 5. IGF-II, 10 nM, EphB4 promoter −7/−1028; 6.10% FBS, EphB4 promoter −7/−1028; 7. Serum Free, EphB4 promoter −7/−1508mut; 8. IGF-II, 10 nM, EphB4 promoter −7/−1508mut; and 9.10% FBS, EphB4 promoter −7/−1508mut.

FIG. 3 demonstrates that IGF-II induced activation of EphB4 gene expression is mediated by activation of either the IGF-IR or the InsR isoform A at the cellular level. In FIG. 3, Series 1: Firefly Luciferare signal (pGL3-Luc −1508/−7 EphB4 construct); Series 2: Renilla Luciferase signal (mock vector); and RLU=Relative Luciferase Units. Conditions: 1. R⁺ cells (IGF-IR⁺); Serum Free, EphB4 promoter −7/−1508; 2. R⁺ cells (IGF-IR⁺); IGF-II, 10 nM, EphB4 promoter −7/−1508; 3. R-IRA cells (InsR-A⁺); Serum Free, EphB4 promoter −7/−1508; 4. R-IRA cells (InsR-A⁺); IGF-II, 10 nM, EphB4 promoter −7/−1508.

FIG. 4 demonstrate that EphB4 is ectopically expressed in IGF-II positive cancer cell lines. EphB4 expressing cell lines that previously tested positive for expression include the following: 1. FF1; human Thyroid anaplastic carcinoma cell line; 2. ARO1; human Thyroid anaplastic carcinoma cell line; 3. 8305c; human Thyroid anaplastic carcinoma cell line; 4. stromal tissue (retroperitoneal); 5. LNCaP; human Prostate carcinoma cell line; 6. PC3; human Prostate carcinoma cell line; 7. Normal skin (subcutaneous); 8. 293; human kidney carcinoma cell line; 9. MCF7; human breast carcinoma cell line; 10. KS1; human kaposi sarcoma cell line; 11. Hela; human Cervical Carcinoma cell line; 12. SaOS; human OsteoSarcoma cell line; 13. Patient 1; primary bone cancer; 14. Patient 2; lymph node metastasis; 15. Patient 3; primary bone cancer; Patient 4; liver metastasis; and 17. Patient 5; primary bone cancer.

### DETAILED DESCRIPTION OF THE INVENTION

### Introduction

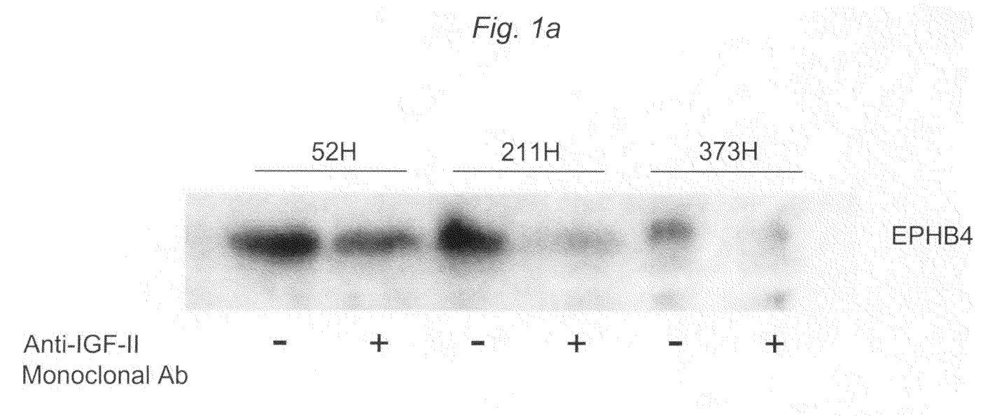

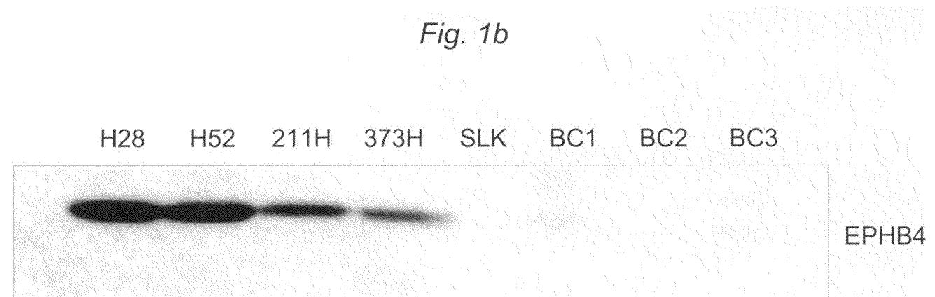

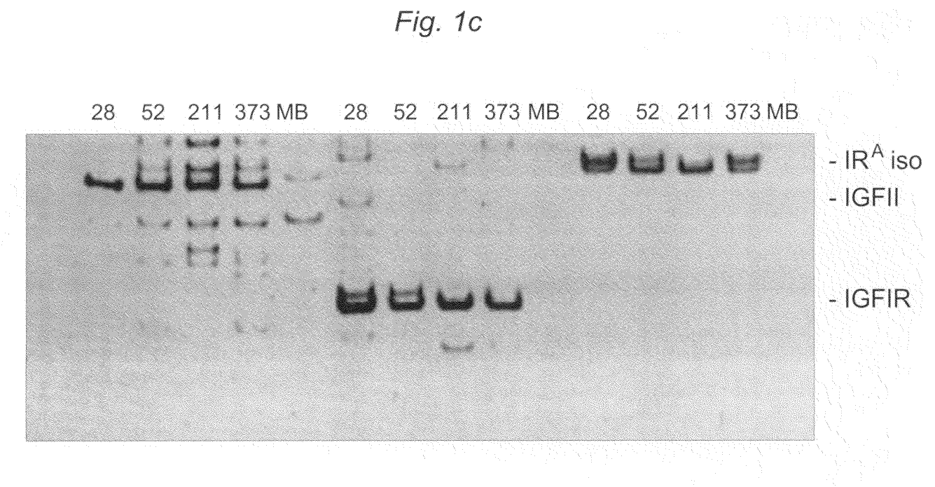

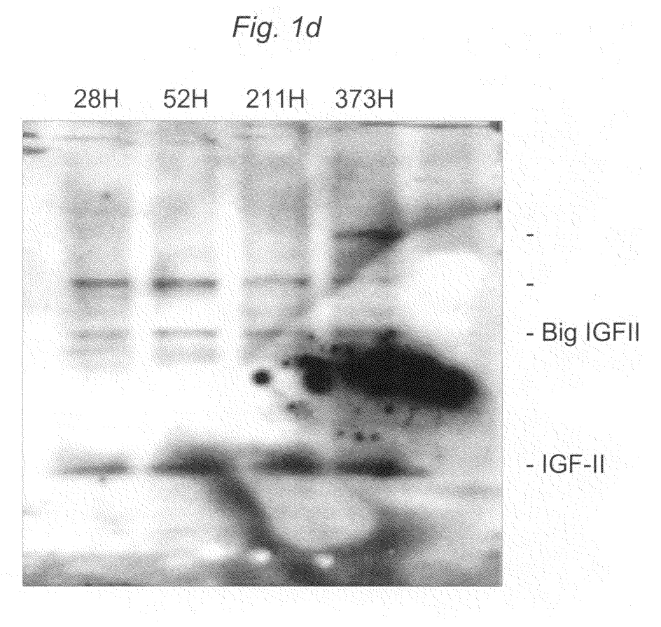

It has now been discovered that cancer-secreted IGF-II is responsible for a self-stimulatory loop resulting in the up-regulation of the EphB4 protein. The discovery of this self-stimulatory mechanism is based, in part, on the finding that both the IGF-IR and the InsR-A along with IGF-II messenger RNA and secreted protein are expressed in the same cells in four different Mesothelioma cell lines (see, Example 1 and FIGS. 1C and 1D). Further evidence of such functional relationship between IGF-II and EphB4 is shown herein where an IGF-II neutralizing antibody added to three distinct Mesothelioma cancer cell lines in culture was able to dramatically decrease the EphB4 protein levels. This novel observation re-evaluates the role of IGF-II and discloses a new mechanism by which IGF-II can favor blood vessel formation in cancer and cause tumor progression from a small and self-contained tumor mass up to a more vascularized and fast-growing tumor.

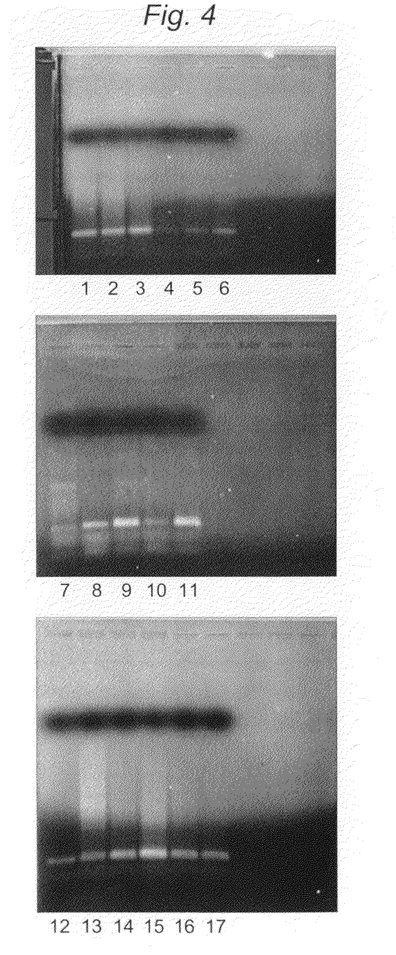

The findings of the present invention further support earlier findings relating to the ectopic expression of the venous marker EphB4 in head and neck cancers (Sinha et al., Ear Nose Throat J., 82(11):866-887 (2003)), which has also been shown in other type of cancers (Xia et al., Clin. Cancer Res., 11(12):4305-4315 (2005); and Xia et al., Cancer Res., 65(11):4623-4632 (2005); Wu et al., Oncology Research, 10(1):26-33 (2004)). To further establish the causative role of IGF-II-dependent EphB4 expression in cancer, embryologically distinct cancer cell lines that had previously tested positive for IGF-II expression were screened for EphB4 expression, and a strong correlation was found to exist between IGF-II expression and EphB4 expression (see, Example 4 and FIG. 4).

The functional relationship between IGF-II and EphB4 has also been demonstrated at the gene expression level (see, Example 2). By using three different gene-reporter constructs that contain regions −1508 to −7, −1028 to −7 and a mutant of the first construct (i.e., −1508 to −7) containing a mutation at position −1365, which has previously been shown to be essential for the regulation of the EphB4 gene (Bruhl et al., Circulation Res. 94:743-751 (2004)), it has been demonstrated that IGF-II-induced signaling, which is either through activation of the IGF-IR or via the InsR-A receptor, causes the activation of the EphB4 reporter gene through its “full length” promoter region, but not when the −1500 to −1028 region is deleted or when the human EphB4 promoter is mutated at position −1365 containing a TAAT motif targeted by the Hox9 transcription factor (Bruhl et al., supra). These findings are direct proof that the IGF-II control of EphB4 expression occurs at the gene transcriptional level and are further proof that the signaling pathway leading to this effect is highly conserved in mammalian cells (mouse to human). These findings are also direct proof that the transcription factor HoxA9 and/or its cancer expressed orthologs and/or paralogs acting on the TAAT motif at position −1365 of the human EphB4 promoter is/are directly involved in mediating the IGF-II stimulatory effect on EPHB4 and that methods targeting Hox binding to the TAAT motif at position −1365 of the human EphB4 promoter can be used to inhibit the IGF-II effect of EphB4 in cancer.

In view of the above remarkable findings, the present invention provides, in part, diagnostic methods for qualifying tumor status (e.g., benign tumor growth, transitional tumor growth or malignant tumor growth) in a subject, screening methods for identifying compounds that modulate (e.g., inhibits) angiogenesis in a cell that co-expresses IGF-II and EphB4 or in a cell wherein EphB4 expression is driven by IGF-II expression in the surrounding parenchymal or stromal tissue (e.g., fibroblasts); screening methods for identifying compounds that inhibit tumorgenesis in a tumor cell that co-expresses IGF-II and EphB4, methods of modulating angiogenesis in a cell that co-expresses IGF-II and EphB4 or in a cell wherein EphB4 expression is driven by IGF-II expression in the surrounding stromal or parenchymal tissue (e.g., fibroblasts), methods of inhibiting tumorigenesis in a tumor cell that co-expresses IGF-II and EphB4 or in a tumor cell wherein EphB4 expression is driven by IGF-II expression in the surrounding stromal or parenchymal tissue (e.g., fibroblasts), EphB4-specific targeting compositions, EphB4-specific targeting vehicles as well as diagnostic kits for qualifying tumor status in a subject.

### DEFINITIONS

By “disorder associated with angiogenesis or tumorigenesis” or “disease associated with angiogenesis or tumorigenesis” herein is meant a disease state which is marked by an excess of vessel development. Angiogenesis and tumorigenesis disorders associated with increased angiogenesis include, but are not limited to, breast, lung, colon, ovarian, liver, stomach, bladder, thyroid, and prostate cancer, basal cell carcinoma, melanoma, lymphomas, leukemias, e.g., myeloid leukemia (AML, CML), endometriosis, diabetic retinopathy, glaucoma, glomerulonephritis, rheumatoid arthritis, and age related macular degeneration.

By “disorder associated with cellular proliferation or tumorigenesis” or “disease associated with cellular proliferation or tumorigenesis” herein is meant a disease state which is marked by an excess of cellular proliferation or apoptosis. Such disorders associated with increased cellular proliferation and include, but are not limited to, cancer and non-cancerous pathological proliferation.

The terms “IGF-II polypeptide” or a nucleic acid encoding an “IGF-II polypeptide”, “IGF-II TK receptor polypeptide” or a nucleic acid encoding an “IGF-II TK receptor polypeptide” and “EphB4 polypeptide” or a nucleic acid sequence encoding an “EphB4 polypeptide” refer to nucleic acid and polypeptide polymorphic variants, alleles, mutants, and interspecies homologs that: (1) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of over a region of at least about 25, 50, 100, 200, 500, 1000, or more amino acids, to a polypeptide encoded by a referenced nucleic acid or an amino acid sequence, such as those disclosed in Bell et al., Nature, 310(5980):775-77 (1984) for IGF-II, in Bennet et al., J. Biol. Chem., 269(19):14211-14218 (1994) for EphB4, in Ullrich et al., EMBO J., 5:2503-2512 (1986) for IGF-IR and in Ulrich et al., Nature, 313:756-761 (1985) and Seino et al., Biochem. Biophys. Res. Commun., 159(1):312-316 (1989) for InsR-A; (2) specifically bind to antibodies, e.g., polyclonal antibodies, raised against an immunogen comprising a referenced amino acid sequence, immunogenic fragments thereof, and conservatively modified variants thereof; (3) specifically hybridize under stringent hybridization conditions to a nucleic acid encoding a referenced amino acid sequence, and conservatively modified variants thereof; (4) have a nucleic acid sequence that has greater than about 95%, preferably greater than about 96%, 97%, 98%, 99%, or higher nucleotide sequence identity, preferably over a region of at least about 25, 50, 100, 200, 500, 1000, or more nucleotides, to a reference nucleic acid sequence. A polynucleotide or polypeptide sequence is typically from a mammal including, but not limited to, primate, e.g., human; rodent, e.g., rat, mouse, hamster; cow, pig, horse, sheep, or any mammal. The nucleic acids and proteins of the invention include both naturally occurring or recombinant molecules.

The phrase “functional effects” in the context of assays for testing compounds that modulate activity of IGF-II and, in turn, EphB4 includes the determination of a parameter that is indirectly or directly under the influence of IGF-II and/or EphB4, e.g., a chemical or phenotypic effect such as loss-of angiogenesis or tumorigenesis phenotype represented by a change in expression of a cell surface marker αvβ3 integrin, changes in cellular migration, changes in endothelial tube formation, and changes in tumor growth, or changes in cellular proliferation, especially endothelial cell proliferation; or enzymatic activity; or, e.g., a physical effect such as ligand binding or inhibition of ligand binding. A functional effect therefore includes ligand binding activity, the ability of cells to proliferate, expression in cells undergoing angiogenesis or tumorigenesis, and other characteristics of angiogenic and tumorigenic cells. “Functional effects” include in vitro, in vivo, and ex vivo activities.

By “determining the functional effect” is meant assaying for a compound that increases or decreases a parameter that is indirectly or directly under the influence of IGF-II and/or EphB4, e.g., measuring physical and chemical or phenotypic effects. Such functional effects can be measured by any means known to those skilled in the art, e.g., changes in spectroscopic characteristics (e.g., fluorescence, absorbance, refractive index); hydrodynamic (e.g., shape), chromatographic; or solubility properties for the protein; ligand binding assays, e.g., binding to antibodies; measuring inducible markers or transcriptional activation of IGF-II and/or EphB4; measuring changes in enzymatic activity; the ability to increase or decrease cellular proliferation, apoptosis, cell cycle arrest, measuring changes in cell surface markers, e.g., αvβ3 integrin; and measuring cellular proliferation, particularly endothelial cell proliferation. Determination of the functional effect of a compound on angiogenesis or tumorigenesis can also be performed using assays known to those of skill in the art such as endothelial cell tube formation assays; haptotaxis assays; the chick CAM assay; the mouse corneal assay; VEGF receptor assays, co-culture tube formation assays, and assays that assess vascularization of an implanted tumor. Tumorigenesis can be measured using in vivo mouse models such as a xenograft model. The functional effects can be evaluated by many means known to those skilled in the art, e.g., microscopy for quantitative or qualitative measures of alterations in morphological features, e.g., tube or blood vessel formation, measurement of changes in RNA or protein levels for IGF-II and/or EphB4, measurement of RNA stability, identification of downstream or reporter gene expression (CAT, luciferase, β-gal, GFP and the like), e.g., via chemiluminescence, fluorescence, colorimetric reactions, antibody binding, inducible markers, etc.

“Inhibitors” and “modulators” of IGF-II polynucleotide and polypeptide sequences are used to refer to inhibitory or modulating molecules identified using in vitro and in vivo assays of IGF-II polynucleotide and polypeptide sequences. Inhibitors are compounds that, e.g., bind to, partially or totally block activity, decrease, prevent, delay activation, inactivate, desensitize, or down regulate the activity or expression of angiogenesis and tumorigenesis proteins, e.g., antagonists. Inhibitors or modulators also include genetically modified versions of IGF-II and/or EphB4, e.g., versions with altered activity, as well as naturally occurring and synthetic ligands, antagonists, agonists, antibodies, peptides, cyclic peptides, nucleic acids, antisense molecules, ribozymes, RNAi molecules, small organic molecules and the like. Such assays for inhibitors include, e.g., expressing IGF-II and/or EphB4 protein in vitro, in cells, or cell extracts, applying putative modulator compounds, and then determining the functional effects on activity, as described above.

Samples or assays comprising IGF-II and/or EphB4 proteins that are treated with a potential inhibitor or modulator are compared to control samples without the inhibitor or modulator to examine the extent of inhibition. Control samples (untreated with inhibitors) are assigned a relative protein activity value of 100%. Inhibition of IGF-II, for example, is achieved when the activity value relative to the control is about 80%, preferably 50%, more preferably 25-0%. Similarly, inhibition of EphB4, for example, is achieved when the activity value relative to the control is about 80%, preferably 50%, more preferably 25-0%.

The term “test compound” or “drug candidate” or “modulator” or grammatical equivalents, as used herein, describes any molecule, either naturally occurring or synthetic, e.g., protein, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, peptide, circular peptide, lipid, fatty acid, siRNA, polynucleotide, oligonucleotide, etc., to be tested for the capacity to directly or indirectly modulate angiogenesis and tumorigenesis. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

A “small organic molecule” refers to an organic molecule, either naturally occurring or synthetic, that has a molecular weight of more than about 50 daltons and less than about 2500 daltons, preferably less than about 2000 daltons, preferably between about 100 to about 1000 daltons, more preferably between about 200 to about 500 daltons.

“RNAi molecule” or an “siRNA” refers to a nucleic acid that forms a double stranded RNA, which double stranded RNA has the ability to reduce or inhibit expression of a gene or target gene when the siRNA expressed in the same cell as the gene or target gene. “siRNA” thus refers to the double stranded RNA formed by the complementary strands. The complementary portions of the siRNA that hybridize to form the double stranded molecule typically have substantial or complete identity. In one embodiment, an siRNA refers to a nucleic acid that has substantial or complete identity to a target gene and forms a double stranded siRNA. The sequence of the siRNA can correspond to the full length target gene, or a subsequence thereof. Typically, the siRNA is at least about 15-50 nucleotides in length (e.g., each complementary sequence of the double stranded siRNA is 15-50 nucleotides in length, and the double stranded siRNA is about 15-50 base pairs in length, preferable about preferably about 20-30 base nucleotides, preferably about 20-25 nucleotides in length, e.g., 20, 21, 22, 23, 24, 25, 26, 27, 28, 29, or 30 nucleotides in length.

“Biological sample” include sections of tissues such as biopsy and autopsy samples, and frozen sections taken for histologic purposes. Such samples include blood, sputum, tissue, cultured cells, e.g., primary cultures, explants, and transformed cells, stool, urine, etc. A biological sample is typically obtained from a eukaryotic organism, most preferably a mammal such as a primate, e.g., chimpanzee or human; cow; dog; cat; a rodent, e.g., guinea pig, rat, mouse; rabbit; or a bird; reptile; or fish.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 70% identity, preferably 75%, 80%, 85%, 90%, 91%, 92%, 93%, 94%, 95%, 96%, 97%, 98%, 99%, or higher identity over a specified region (such as, e.g., those disclosed in Bell et al., Nature, 310(5980):775-77 (1984) for IGF-H, in Bennet et al., J. Biol. Chem., 269(19):14211-14218 (1994) for EphB4, in Ullrich et al., EMBO J., 5:2503-2512 (1986) for IGF-IR and in Ulrich et al., Nature, 313:756-761 (1985) and Seino et al., Biochem. Biophys. Res. Commun., 159(1):312-316 (1989) for InsR-A), when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA, 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res., 25:3389-3402 (1977) and Altschul et al., J. Mol. Biol., 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA, 89:10915 (1989)) alignments (B) of 50, expectation (E) of 10, M=5, N=−4, and a comparison of both strands.

“Nucleic acid” refers to deoxyribonucleotides or ribonucleotides and polymers thereof in single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)). The term nucleic acid is used interchangeably with gene, cDNA, mRNA, oligonucleotide, and polynucleotide.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3^{rd} ed. 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains, e.g., transmembrane domains, pore domains, and cytoplasmic tail domains. Domains are portions of a polypeptide that form a compact unit of the polypeptide and are typically 15 to 350 amino acids long. Exemplary domains include domains with enzymatic activity, e.g., ligand binding domains, etc. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel et al.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)'₂, a dimer of Fab which itself is a light chain joined to V_{H}—C_{H}1 by a disulfide bond. The F(ab)'₂ may be reduced under mild conditions to break the disulfide linkage in the hinge region, thereby converting the F(ab)'₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature, 348:552-554 (1990))

For preparation of antibodies, e.g., recombinant, monoclonal, or polyclonal antibodies, many technique known in the art can be used (see, e.g., Kohler & Milstein, Nature 256:495-497 (1975); Kozbor et al., Immunology Today, 4:72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (U.S. Pat. No. 4,946,778, U.S. Pat. No. 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology, 10:779-783 (1992); Lonberg et al., Nature, 368:856-859 (1994); Morrison, Nature, 368:812-13 (1994); Fishwild et al., Nature Biotechnology, 14:845-51 (1996); Neuberger, Nature Biotechnology, 14:826 (1996); and Lonberg & Huszar, Intern. Rev. Immunol., 13:65-93 (1995)). Alternatively, phage display technology can be used to identify antibodies and heteromeric Fab fragments that specifically bind to selected antigens (see, e.g., McCafferty et al., Nature, 348:552-554 (1990); Marks et al., Biotechnology, 10:779-783 (1992)). Antibodies can also be made bispecific, i.e., able to recognize two different antigens (see, e.g., WO 93/08829, Traunecker et al., EMBO J., 10:3655-3659 (1991); and Suresh et al., Methods in Enzymology, 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Jones et al., Nature, 321:522-525 (1986); Riechmann et al., Nature, 332:323-327 (1988); Verhoeyen et al., Science, 239:1534-1536 (1988) and Presta, Curr. Op. Struct. Biol., 2:593-596 (1992)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

The antibodies of the present invention can be conjugated to an “effector” moiety. The effector moiety can be any number of molecules, including labeling moieties such as radioactive labels or fluorescent labels, or the effector moiety can be a therapeutic moiety. In one aspect, the antibody modulates the activity of IGF-II and, in turn, EphB4.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies, polymorphic variants, alleles, orthologs, and conservatively modified variants, or splice variants, or portions thereof, can be selected to obtain only those polyclonal antibodies that are specifically immunoreactive with angiogenesis proteins and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

### Diagnostic Methods for Qualifying Tumor Status

In one aspect, the present invention provides methods for qualifying tumor status in a subject. Qualifying or determining tumor status typically involves classifying an individual into one of two or more groups (statuses) based on the results of the diagnostic test. The diagnostic tests described herein can be used to classify between a number of different tumor states, i.e., benign tumor growth, transitional tumor growth and malignant tumor growth.

Thus, the present invention provides methods for determining the tumor status in a subject (status: benign, transitional or malignant). Tumor status is determined by detecting/measuring the presence or absence of IGF-II, a IGF-II TK receptor (such as IGF-IR and/or InsR-A) and EphB4 in a biological sample from a subject, and then either submitting them to a classification algorithm or comparing them with a reference amount and/or pattern of IGF-II, the IGF-II TK receptor and EphB4 that is associated with the particular tumor status. In a preferred embodiment, the biological sample is a tumor cell.

In one embodiment, the tumor status is benign and the biological sample is IGF-II negative/IGF-II TK receptor positive (i.e., IGF-IR and/or InsR-A positive)/EphB4 negative. In another embodiment, the tumor status is transitional and the biological sample is IGF-II negative/IGF-II TK receptor positive/EphB4 positive. In this embodiment, the method further comprises detecting the presence or absence of IGF-II in stromal or parenchymal tissue surrounding the tumor, wherein the presence of IGF-II in surrounding stromal or parenchymal tissue along with the presence of its TK-receptors (IGF-1R and/or InsR-A) within the tumor further indicates that the tumor status is transitional. In yet another embodiment, the tumor status is malignant and the biological sample is IGF-II positive/IGF-II TK receptor positive/EphB4 positive.

In one embodiment of the diagnostic methods of the present invention, IGF-II, the IGF-II TK receptor and EphB4 are detected or measured by immunoassay. Immunoassay requires biospecific capture reagents, such as antibodies, to capture IGF-II, the IGF-II TK receptor and EphB4. As described herein, antibodies that specifically bind IGF-II, the IGF-II TK receptor (e.g., IGF-IR or InsR-A) and EphB4 can be produced by methods well known in the art, e.g., by immunizing animals with IGF-II, the IGF-II TK receptor and EphB4. IGF-II, the IGF-II TK receptor and EphB4 can be isolated from samples based on their binding characteristics. Alternatively, since the amino acid sequences of IGF-II, the IGF-II TK receptors (i.e., IGF-IR and InsR-A) and EphB4 are known, the polypeptides can be synthesized and used to generate antibodies by methods well known in the art. For example, MAB clone 292, which is commercially available from R & D Systems Inc. (Minneapolis, Minn.), can be used to detect IGF-II, MAB clone alpha-IR3, which is commercially available from Calbiochem-EMD Chemicals Inc. (San Diego, Calif.), can be used to detect IGF-IR, clone MA-20, which is commercially available from Novus Biologicals Inc. (Littleton, Colo.), can be used to detect InsR-A; and PAB 5536, which is commercially available from Santa Cruz Biotechnology (Santa Cruz, Calif.), can be used to detect EphB4.

This invention contemplates traditional immunoassays including, for example, sandwich immunoassays including ELISA or fluorescence-based immunoassays, as well as other enzyme immunoassays. Nephelometry is an assay done in liquid phase, in which antibodies are in solution. Binding of the antigen to the antibody results in changes in absorbance, which is then measured. As set forth below, in a SELDI-based immunoassay, a biospecific capture reagent for IGF-II and/or IGF-IR and/or InsR-A or EphB4 is attached to the surface of an MS probe, such as a pre-activated ProteinChip array. The biomarker is then specifically captured on the biochip through this reagent, and the captured biomarker is detected by mass spectrometry.

In another embodiment, the IGF-II, the IGF-II TK receptor (e.g., IGF-IR and/or InsR-A) and EphB4 biomarkers of this invention are detected by mass spectrometry, a method that employs a mass spectrometer to detect gas phase ions. Examples of mass spectrometers are time-of-flight, magnetic sector, quadrupole filter, ion trap, ion cyclotron resonance, electrostatic sector analyzer and hybrids of these.

In a preferred method, the mass spectrometer is a laser desorption/ionization mass spectrometer. In laser desorption/ionization mass spectrometry, the analytes are placed on the surface of a mass spectrometry probe, a device adapted to engage a probe interface of the mass spectrometer and to present an analyte to ionizing energy for ionization and introduction into a mass spectrometer. A laser desorption mass spectrometer employs laser energy, typically from an ultraviolet laser, but also from an infrared laser, to desorb analytes from a surface, to volatilize and ionize them and make them available to the ion optics of the mass spectrometer. The analysis of proteins by LDI can take the form of MALDI or of SELDI.

Laser desorption/ionization in a single TOF instrument typically is performed in linear extraction mode. Tandem mass spectrometers can employ orthogonal extraction modes.

In another aspect, this invention provides methods for determining the risk of developing disease (e.g., cancer) in a subject. IGF-II, IGF-II TK receptor and EphB4 amounts or patterns are characteristic of various risk states, e.g., high, medium or low. For instance, the risk of developing a malignant tumor is determined by detecting/measuring IGF-II, the IGF-II TK receptor and EphB4, and then either submitting them to a classification algorithm or comparing them with a reference amount and/or pattern of IGF-II, the IGF-II TK receptor and EphB4 that is associated with the particular risk level.

In another aspect, the present invention provides methods for determining the stage of tumor growth in a subject. Each stage of the tumor growth has a characteristic amount of IGF-II, IGF-II TK receptor and EphB4 or a relative amount of IGF-II, IGF-II TK receptor and EphB4 (a pattern). The stage of tumor growth is determined by detecting/measuring IGF-II, the IGF-II TK receptor and EphB4, and then either submitting them to a classification algorithm or comparing them with a reference amount and/or pattern of IGF-II, the IGF-II TK receptor and EphB4 that is associated with the particular stage of tumor growth. For example, one can classify between benign tumor growth, transitional tumor growth and malignant tumor growth based on the presence or absence of IGF-II, the IGF-II TK receptor (i.e., IGF-IR and/or InsR-A) and EphB4.

In another aspect, this invention provides methods for determining the course of disease in a subject. Disease course refers to changes in disease status over time, including disease progression (worsening) and disease regression (improvement). Over time, the amounts or relative amounts (e.g., the pattern) of IGF-II and EphB4 changes. Therefore, the trend of these markers, either increased or decreased, over time toward diseased or non-diseased indicates the course of the disease. Accordingly, this method involves measuring IGF-II and EphB4 in a subject at least two different time points, e.g., a first time and a second time, and comparing the change in amounts, if any. The course of disease is determined based on these comparisons.

Similarly, changes in the rate of disease progression (or regression) may be monitored by measuring the amount of IGF-II, the IGF-II TK receptor and EphB4 at different times and calculating the rate of change in IGF-II, the IGF-II TK receptor and EphB4 levels. The ability to measure disease state or velocity of disease progression can be important for drug treatment studies where the goal is to slow down or arrest disease progression through therapy.

In connection with the diagnostic methods of the present invention, additional embodiments relate to the communication of assay results or diagnoses or both to technicians, physicians or patients, for example. In certain embodiments, computers will be used to communicate assay results or diagnoses or both to interested parties, e.g., physicians and their patients. In some embodiments, the assays will be performed or the assay results analyzed in a country or jurisdiction that differs from the country or jurisdiction to which the results or diagnoses are communicated.

In a preferred embodiment of the invention, a diagnosis based on the differential presence in a test subject of IGF-II, the IGF-II TK receptor (such as IGF-IR and/or InsR-A) and EphB4 is communicated to the subject as soon as possible after the diagnosis is obtained. The diagnosis may be communicated to the subject by the subject's treating physician. Alternatively, the diagnosis may be sent to a test subject by e-mail or communicated to the subject by phone. A computer may be used to communicate the diagnosis by e-mail or phone. In certain embodiments, the message containing results of a diagnostic test may be generated and delivered automatically to the subject using a combination of computer hardware and software which will be familiar to artisans skilled in telecommunications. One example of a healthcare-oriented communications system is described in U.S. Pat. No. 6,283,761; however, the present invention is not limited to methods which utilize this particular communications system. In certain embodiments of the methods of the invention, all or some of the method steps, including the assaying of samples, diagnosing of diseases, and communicating of assay results or diagnoses, may be carried out in diverse (e.g., foreign) jurisdictions.

In certain embodiments of the methods of qualifying tumor status, the methods further comprise managing subject treatment based on the status. Such management includes the actions of the physician or clinician subsequent to determining tumor status. For example, if a physician makes a diagnosis of malignant tumor, then a certain regime of treatment, such as prescription or administration of an anti-chemotherapeutic agent, might follow. Alternatively, a diagnosis of benign tumor might be followed with further testing to determine a specific disease the patient might be suffering from. Also, if the diagnostic test gives an inconclusive result on tumor status, further tests may be called for.

Screening Methods for Identifying Modulators of Angiogenesis and/or Tumorigenesis

As explained herein, it has now been discovered that EphB4, which is a venous marker that is involved in blood vessel formation, is under the direct regulation and control of IGF-II. It has further been discovered that the inhibition, i.e., blocking, of IGF-II results in the inhibition or blocking of EphB4 gene or protein expression which, in turn, results in the inhibition of angiogenesis and proliferation. Thus, the present invention provides screening methods for identifying compounds that modulate angiogenesis in a cell that co-expresses IGF-II and EphB4 or in a cell wherein EphB4 expression is driven by IGF-II expression in the surrounding parenchymal or stromal tissue (e.g., fibroblasts). Similarly, the present invention provides screening methods for identifying compounds that inhibit tumorigenesis in a tumor cell that co-expresses IGF-II and EphB4 or in a tumor cell wherein EphB4 expression is driven by IGF-II expression in the surrounding parenchymal or stromal tissue (e.g., fibroblasts). Examples of such tumor cells include, but are not limited to those, set forth in FIG. 4 and include, for example, breast, thyroid, liver, prostrate, kidney, bone, lymph node, cervix, colon, skin, osteosarcoma, etc.

Typically, screening a test compound includes obtaining samples (e.g., biopsy of a solid tumor) from test subjects before and after the subjects have been exposed to a test compound. The levels in the samples of IGF-II, the IGF-II TK receptor and/or EphB4 may be measured and analyzed to determine whether the levels of these biomarkers change after exposure to a test compound. The samples can be analyzed by any appropriate means known to one of skill in the art, such as by immunoassay or mass spectrometry. For example, the levels of IGF-II and/or the IGF-II TK receptor and/or EphB4 may be measured directly by Western blot using radio- or fluorescently-labeled antibodies which specifically bind to IGF-II, the IGF-II TK receptor or EphB4. Alternatively, changes in the levels of mRNA encoding IGF-II and/or IGF-II TK receptor and/or EphB4 may be measured and correlated with the administration of a given test compound to a subject. In a further embodiment, the changes in the level of expression of IGF-II and/or the IGF-II TK receptor and/or EphB4 may be measured using in vitro methods and materials. For example, human tissue cultured cells which express, or are capable of expressing, IGF-II and/or IGF-II TK receptor and/or EphB4 may be contacted with test compounds. Subjects who have been treated with test compounds will be routinely examined for any physiological effects which may result from the treatment. In particular, the test compounds will be evaluated for their ability to inhibit angiogenesis and to decrease disease likelihood in a subject. Alternatively, if the test compounds are administered to subjects who have previously been diagnosed with cancer, test compounds will be screened for their ability to slow or stop the progression of the disease.

Following are exemplary assays that can be used to screen a compound for its ability to inhibit IGF-II and, in turn, EphB4. Again, compounds found to inhibit IGF-II can be used to inhibit angiogenesis and/or tumorigenesis. Those of skill in the art will appreciate that in addition to the assays disclosed herein, numerous other assays can be used to screen a compound for its ability to inhibit IGF-II and, in turn, EphB4.

A. Assays

Modulation of IGF-II and, in turn, EphB4, which results in the corresponding modulation of angiogenesis or tumorigenesis, can be assessed using a variety of in vitro and in vivo assays, including high throughput ligand binding and cell based assays, as described herein. Such assays can be used to test for inhibitors of IGF-II and/or EphB4, and, consequently, inhibitors of angiogenesis or tumorigenesis. Such modulators of IGF-II and, in turn, EphB4 are useful for treating angiogenic and tumorigenic disorders. Modulators of IGF-II are tested using either recombinant or naturally occurring protein, preferably human protein.

Measurement of an angiogenic or tumorigenic or loss-of-angiogenesis or tumorigenesis phenotype on the protein (e.g., IGF-II) or cell expressing the protein (e.g., IGF-II or IGF-II and EphB4), either recombinant or naturally occurring, can be performed using a variety of assays, in vitro, in vivo, and ex vivo. For example, recombinant or naturally occurring protein can be used in vitro, in a ligand binding or enzymatic function assay. Protein present in a cellular extract can also be used in in vitro assays. Cell- and animal-based in vivo assays can also be used to assay for modulators of angiogenesis. Any suitable physical, chemical, or phenotypic change that affects activity or binding can be used to assess the influence of a test compound on IGF-II and, in turn, EphB4. When the functional effects are determined using intact cells or animals, one can also measure a variety of effects such as, in the case of angiogenesis associated with tumors, tumor growth, neovascularization, endothelial tube formation, cell surface markers such as αvβ3, hormone release, transcriptional changes to both known and uncharacterized genetic markers (e.g., northern blots), changes in cell metabolism such as cell growth or pH changes, and changes in intracellular second messengers such as cGMP. In one embodiment, measurement of αvβ3 integrin cell surface expression and FACS sorting is used to identify modulators of angiogenesis.

In Vitro Assays

Assays to identify compounds that inhibit or block IGF-II, i.e., compounds with angiogenesis or tumorigenesis modulating activity (or anti-angiogenic or anti-tumorigenic activity), can be performed in vitro, e.g., binding assays. Purified recombinant or naturally occurring protein, such as IGF-II, can be used in the in vitro methods of the invention. In addition to purified protein, the recombinant or naturally occurring protein, such as IGF-II, can be part of a cellular lysate. As described herein, the assay can be either solid state or soluble. Preferably, the protein is bound to a solid support, either covalently or non-covalently. Often, the in vitro assays of the invention are ligand binding or ligand affinity assays, either non-competitive or competitive. Other in vitro assays include measuring changes in spectroscopic (e.g., fluorescence, absorbance, refractive index), hydrodynamic (e.g., shape), chromatographic, or solubility properties for the protein.

In one embodiment, a high throughput binding assay is performed in which the protein or chimera comprising a fragment thereof is contacted with a potential modulator and incubated for a suitable amount of time. In one embodiment, the potential modulator is bound to a solid support, and the protein is added. In another embodiment, the protein is bound to a solid support. A wide variety of modulators can be used, as described below, including small organic molecules, peptides, siRNA and antibodies. A wide variety of assays can be used to identify angiogenesis-modulator binding, including labeled protein-protein binding assays, electrophoretic mobility shifts, immunoassays, and the like. In some cases, the binding of the candidate modulator is determined through the use of competitive binding assays, where interference with binding of a known ligand is measured in the presence of a potential modulator. Often, either the potential modulator or the known ligand is labeled.

Cell-Based In Vivo Assays

In another embodiment, the protein is expressed in a cell, and functional, e.g., physical and chemical or phenotypic, changes are assayed to identify angiogenesis or tumorigenesis modulators, preferably anti-angiogenesis or anti-tumorigenesis compounds. Cells expressing IGF-II (and preferably EphB4) can also be used in binding assays or enzymatic assays. Any suitable functional effect can be measured, as described herein. For example, ligand binding, cell surface marker expression, cellular proliferation, VEGF-R assays, co-culture assays for tube formation, and cell migration assays are all suitable assays to identify potential modulators using a cell based system. Suitable cells for such cell based assays include both primary endothelial cells and cell lines, as described herein. The protein can be naturally occurring or recombinant.

As described above, in one embodiment, loss-of angiogenesis or tumorigenesis phenotype is measured by contacting endothelial cells comprising IGF-II with a potential modulator. Modulation of EphB4 and, in turn, angiogenesis or tumorigenesis is identified by screening for cell surface marker expression, e.g., αvβ3 integrin expression levels, using fluorescent antibodies and FACS sorting.

In another embodiment, cellular proliferation can be measured using ³H-thymidine incorporation or dye inclusion.

In another embodiment, cellular polypeptide levels (e.g., evels of IGF-II and EphB4) are determined by measuring the level of protein or mRNA. The level of protein or proteins are measured using immunoassays such as western blotting, ELISA and the like with an antibody that selectively binds to the polypeptide or a fragment thereof. For measurement of mRNA, amplification, e.g., using PCR, LCR, or hybridization assays, e.g., northern hybridization, RNAse protection, dot blotting, are preferred. The level of protein or mRNA is detected using directly or indirectly labeled detection agents, e.g., fluorescently or radioactively labeled nucleic acids, radioactively or enzymatically labeled antibodies, and the like, as described herein.

Alternatively, protein expression can be measured using a reporter gene system. Such a system can be devised using an angiogenesis protein promoter operably linked to a reporter gene such as chloramphenicol acetyltransferase, firefly luciferase, bacterial luciferase, β-galactosidase and alkaline phosphatase. Furthermore, the protein of interest can be used as an indirect reporter via attachment to a second reporter such as red or green fluorescent protein (see, e.g., Mistili & Spector, Nature Biotechnology 15:961-964 (1997)). The reporter construct is typically transfected into a cell. After treatment with a potential modulator, the amount of reporter gene transcription, translation, or activity is measured according to standard techniques known to those of skill in the art.

A variety of phenotypic angiogenesis or tumorigenesis assays are known to those of skill in the art. Various models have been employed to evaluate angiogenesis (e.g., Croix et al., Science, 289:1197-1202 (2000) and Kahn et al., Amer. J. Pathol., 156:1887-1900). Assessment of angiogenesis or tumorigenesis in the presence of a potential modulator can be performed using cell-culture-based assays, e.g., endothelial cell tube formation assays and haptotaxis assays, as well as other animal based bioassays such as the chick CAM assay, the mouse corneal assay, and assays measuring the effect of administering potential modulators on implanted tumors.

For determination of cellular proliferation, any suitable functional effect can be measured, as described herein. For example, cellular morphology (e.g., cell volume, nuclear volume, cell perimeter, and nuclear perimeter), ligand binding, kinase activity, apoptosis, cell surface marker expression, cellular proliferation, GFP positivity and dye dilution assays (e.g., cell tracker assays with dyes that bind to cell membranes), DNA synthesis assays (e.g., ³H-thymidine and fluorescent DNA-binding dyes such as BrdU or Hoescht dye with FACS analysis), G₀/G₁ cell cycle arrest, are all suitable assays to identify potential modulators using a cell based system. Suitable cells for such cell based assays include both primary cancer or tumor cells and cell lines, as described herein in Example 4 (see, FIG. 4).

In one preferred embodiment, a direct quantifiable method to determine the inhibitory effect of any IGF-II blocking compound is the measurement of the level of growth factor-induced activation of its cellular receptors (specifically the level of tyrosine phosphorylation of IGF-IR and InsR-A) that occurs in vivo, followed by capture of the receptors pre-stimulated in vivo following in vitro determination of their activity. This type of assay is, in essence, a combination of a cell-based assay and an in vitro assay as generally described above and typically includes the following general steps:

Step 1. Culturing cell models that express IGF-II tyrosine kinase receptors (e.g., IGF-IR and/or InsR-A) either endogenously or by transient or stable transfection either in presence or absence of fixed or increasing amounts of ligand (e.g., soluble IGF-II either in a highly purified recombinant form or in a secreted form present in conditioned media of IGF-II expressing/secreting cells) and in presence or absence of fixed or increasing amounts of the IGF-II blocking agent. This step is considered a modification of the general method described in Pandini et al., Clin Cancer Res., 5:1935-44 (1999);

Step 2. Generation of Total Cell Lisates (TCLs) that preserve the total phosphorylation that occurred in vivo under the conditions described in Step 1, above, followed by individual capture of an IGF-II receptor of choice (either the IGF-IR and/or the InsR-A) using antibodies raised against the extracellular portion of the receptor (e.g., the alpha-IR3MAB clone for the IGF-IR and the MA-20 MAB clone for the InsR). The immuno-capture of the receptors from a total cell lysate can be performed by incubation of the TCLs with the above antibodies pre-adsobed to ELISA well plates or in form of immunoprecipitation with soluble MABs.

Step 3. ELISA of the captured receptors using an anti-phosphotyrosine antibody (such as the commercially available G410 MAB clone, the commercially available PY20 MAB clone or any other clone having comparable binding affinity) conjugated with a enzymatic system of choice (with or without amplification steps included) for colorimetric/fluorescent/luminescent determination of the receptor phosphorylation in vitro.

Animal Models

A number of animal based assays for angiogenesis or tumorigenesis phenotypes are known to those of skill in the art and can be used to assay for modulators of angiogenesis. For example, the chick CAM assay is described by O'Reilly et al. Cell, 79:315-328 (1994). Briefly, 3 day old chicken embryos with intact yolks are separated from the egg and placed in a petri dish. After 3 days of incubation, a methylcellulose disc containing the protein to be tested is applied to the CAM of individual embryos. After about 48 hours of incubation, the embryos and CAMs are observed to determine whether endothelial growth has been inhibited.

The mouse corneal assay involves implanting a growth factor-containing pellet, along with another pellet containing the suspected endothelial growth inhibitor, in the cornea of a mouse and observing the pattern of capillaries that are elaborated in the cornea.

Angiogenesis can also be measured by determining the extent of neovascularization of a tumor. For example, carcinoma cells can be subcutaneously inoculated into athymic or nude mice or SCID mice and tumor growth then monitored. Immunoassays using endothelial cell-specific antibodies are typically used to stain for vascularization of tumor and the number of vessels in the tumor.

As described above, animal models of angiogenesis find use in screening for modulators of angiogenesis and tumorigenesis. Similarly, transgenic animal technology including gene knockout technology, for example as a result of homologous recombination with an appropriate gene targeting vector, or gene overexpression, will result in the absence or increased expression of the protein. The same technology can also be applied to make knock-out cells. When desired, tissue-specific expression or knockout of the protein may be necessary. Transgenic animals generated by such methods find use as animal models of angiogenesis and are additionally useful in screening for modulators of angiogenesis and tumorigenesis.

Knock-out cells and transgenic mice can be made by insertion of a marker gene or other heterologous gene into the endogenous gene site in the mouse genome via homologous recombination. Such mice can also be made by substituting the endogenous gene with a mutated version of the gene, or by mutating the endogenous gene, e.g., by exposure to carcinogens.

A DNA construct is introduced into the nuclei of embryonic stem cells. Cells containing the newly engineered genetic lesion are injected into a host mouse embryo, which is re-implanted into a recipient female. Some of these embryos develop into chimeric mice that possess germ cells partially derived from the mutant cell line. Therefore, by breeding the chimeric mice it is possible to obtain a new line of mice containing the introduced genetic lesion (see, e.g., Capecchi et al., Science, 244:1288 (1989)). Chimeric targeted mice can be derived according to Hogan et al., Manipulating the Mouse Embryo: A Laboratory Manual, Cold Spring Harbor Laboratory (1988) and Teratocarcinomas and Embryonic Stem Cells: A Practical Approach, Robertson, ed., IRL Press, Washington, D.C., (1987).

B. Modulators

The compounds tested as modulators of IGF-II and, in turn, EphB4 and, in turn, angiogenesis or tumorigenesis can be any small organic molecule, or a biological entity, such as a protein, e.g., an antibody or peptide, a sugar, a nucleic acid, e.g., an antisense oligonucleotide, an RNAi molecule or a ribozyme, or a lipid. Alternatively, modulators can be genetically altered versions of, for example, IGF-II. Typically, test compounds will be small organic molecules, RNAi molecules and antibodies or peptides. Antibodies suitable for use in the present invention include, for example, those known for their IGF-II neutralizing properties, such as the MAB 292 clone, as well as other antibodies with similar properties. Peptides suitable for use in the present invention include, for example, those known to be high affinity IGF-II binders, both the full-length version as well as the truncated versions. Examples of such binding partners include, but are not limited to, IGF Binding Protein (e.g., IGFBP 3 to 7), Mannose 6-phosphate receptor and Vitronectin.

Essentially any chemical compound can be used as a potential modulator or ligand in the assays of the invention. Most often compounds that can be dissolved in aqueous or organic (especially DMSO-based) solutions are used in the screening methods of the present invention. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods involve providing a combinatorial small organic molecule, peptide or siRNA library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res., 37:487-493 (1991) and Houghton et al., Nature, 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA, 90:6909-6913 (1993)), vinylogous polypeptides (Hagihara et al., J. Amer. Chem. Soc., 114:6568 (1992)), nonpeptidal peptidomimetics with glucose scaffolding (Hirschmann et al., J. Amer. Chem. Soc., 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

C. Solid State and Soluble High Throughput Assays

In one embodiment, the invention provides soluble assays using IGF-II and/or a IGF-II TK receptor (e.g., IGF-IR and/or InsR-A) and/or EphB4 or a cell or tissue expressing IGF-II (and/or a IGF-II TK receptor and/or EphB4), either naturally occurring or recombinant. In another embodiment, the invention provides solid phase based in vitro assays in a high throughput format, where IGF-II (and/or a IGF-II TK receptor and/or EphB4) is attached to a solid phase substrate. Any one of the assays described herein can be adapted for high throughput screening, e.g., ligand binding, cellular proliferation, cell surface marker flux, e.g., αvβ3 integrin, etc. In one preferred embodiment, the cell-based system using αvβ3 integrin modulation and FACS assays is used in a high throughput format for identifying modulators of IGF-II and, in turn, EphB4, and therefore modulators of cell angiogenesis and tumorigenesis.

In the high throughput assays of the invention, either soluble or solid state, it is possible to screen up to several thousand different modulators or ligands in a single day. This methodology can be used for angiogenesis proteins, such as IGF-II and/or EphB4, in vitro, or for cell-based assays comprising an angiogenesis protein, such as IGF-II and/or EphB4. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100-about 1500 different compounds. It is possible to assay many plates per day; assay screens for up to about 6,000, 20,000, 50,000, or more than 100,000 different compounds are possible using the integrated systems of the invention.

For a solid state reaction, the protein of interest, such as IGF-II, or a fragment thereof, e.g., an extracellular domain, or a cell comprising the protein of interest, such as IGF-II, or a fragment thereof as part of a fusion protein can be bound to the solid state component, directly or indirectly, via covalent or non covalent linkage e.g., via a tag. The tag can be any of a variety of components. In general, a molecule which binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.) Antibodies to molecules with natural binders such as biotin are also widely available and appropriate tag binders; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherein family, the integrin family, the selectin family, and the like; see, e.g., Pigott & Power, The Adhesion Molecule Facts Book I (1993). Similarly, toxins and venoms, viral epitopes, hormones (e.g., opiates, steroids, etc.), intracellular receptors (e.g. which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly gly sequences of between about 5 and 200 amino acids. Such flexible linkers are known to persons of skill in the art. For example, poly(ethelyne glycol) linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc., 85:2149-2154 (1963) (describing solid phase synthesis of, e.g., peptides); Geysen et al., J. Immun. Meth., 102:259-274 (1987) (describing synthesis of solid phase components on pins); Frank & Doring, Tetrahedron, 44:60316040 (1988) (describing synthesis of various peptide sequences on cellulose disks); Fodor et al., Science, 251:767-777 (1991); Sheldon et al., Clinical Chemistry, 39(4):718-719 (1993); and Kozal et al., Nature Medicine, 2(7):753759 (1996) (all describing arrays of biopolymers fixed to solid substrates). Non-chemical approaches for fixing tag binders to substrates include other common methods, such as heat, cross-linking by UV radiation, and the like.

### Antibodies to IGF-II, IGF-II Tyrosine Kinase Receptors and EphB4 as Well as Uses Thereof

In addition to the detection of gene and gene expression using nucleic acid hybridization technology, one can also use immunoassays to detect IGF-II and EphB4. Such assays are useful in the diagnostic methods of the present invention as well as in the methods for screening for modulators of angiogenesis and tumorigenesis. Immunoassays can be used to qualitatively or quantitatively analyze IGF-II and/or EphB4. A general overview of the applicable technology can be found in Harlow & Lane, Antibodies: A Laboratory Manual (1988).

A. Production of Antibodies

Methods of producing polyclonal and monoclonal antibodies that react specifically with IGF-II and EphB4 are known to those of skill in the art (see, e.g., Coligan, Current Protocols in Immunology (1991); Harlow & Lane, supra; Goding, Monoclonal Antibodies: Principles and Practice (2d ed. 1986); and Kohler & Milstein, Nature, 256:495-497 (1975). Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., Science, 246:1275-1281 (1989); Ward et al., Nature, 341:544-546 (1989)).

A number of immunogens comprising portions of IGF-II and EphB4 may be used to produce antibodies specifically reactive with IGF-II and EphB4. For example, recombinant protein or an antigenic fragment thereof, can be isolated using methods known to those of skill in the art. Recombinant protein can be expressed in eukaryotic or prokaryotic cells using methods known to those of skill, and purified as generally described above. Recombinant protein is the preferred immunogen for the production of monoclonal or polyclonal antibodies. Alternatively, a synthetic peptide derived from IGF-II and/or EphB4 and conjugated to a carrier protein can be used an immunogen. Naturally occurring protein may also be used either in pure or impure form. The product is then injected into an animal capable of producing antibodies. Either monoclonal or polyclonal antibodies may be generated, for subsequent use in immunoassays to measure the protein.

Methods of production of polyclonal antibodies are known to those of skill in the art. An inbred strain of mice (e.g., BALB/C mice) or rabbits is immunized with the protein using a standard adjuvant, such as Freund's adjuvant, and a standard immunization protocol. The animal's immune response to the immunogen preparation is monitored by taking test bleeds and determining the titer of reactivity to the beta subunits. When appropriately high titers of antibody to the immunogen are obtained, blood is collected from the animal and antisera are prepared. Further fractionation of the antisera to enrich for antibodies reactive to the protein can be done if desired (see, Harlow & Lane, supra).

Monoclonal antibodies may be obtained by various techniques familiar to those skilled in the art. Briefly, spleen cells from an animal immunized with a desired antigen are immortalized, commonly by fusion with a myeloma cell (see, Kohler & Milstein, Eur. J. Immunol. 6:511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse et al., Science, 246:1275-1281 (1989).

Monoclonal antibodies and polyclonal sera are collected and titered against the immunogen protein in an immunoassay, for example, a solid phase immunoassay with the immunogen immobilized on a solid support. Typically, polyclonal antisera with a titer of 10⁴ or greater are selected and tested for their cross reactivity against non-angiogenesis proteins, using a competitive binding immunoassay. Specific polyclonal antisera and monoclonal antibodies will usually bind with a K_{d} of at least about 0.1 mM, more usually at least about 1 μM, preferably at least about 0.1 μM or better, and most preferably, 0.01 μM or better. Antibodies specific only for a particular ortholog, such as a human ortholog, can also be made, by subtracting out other cross-reacting orthologs from a species such as a non-human mammal. In this manner, antibodies that bind only to a desired protein may be obtained.

Once the specific antibodies against the protein of interest (e.g., IGF-II or EphB4) are available, the protein can be detected by a variety of immunoassay methods. In addition, the antibody can be used therapeutically as modulators. For a review of immunological and immunoassay procedures, see Basic and Clinical Immunology (Stites & Ten eds., 7^{th} ed. 1991). Moreover, the immunoassays of the present invention can be performed in any of several configurations, which are reviewed extensively in Enzyme Immunoassay (Maggio, ed., 1980); and Harlow & Lane, supra.

B. Immunological Binding Assays

Proteins of interest, such as IGF-II and EphB4, can be detected and/or quantified using any of a number of well recognized immunological binding assays (see, e.g., U.S. Pat. Nos. 4,366,241; 4,376,110; 4,517,288; and 4,837,168). For a review of the general immunoassays, see also Methods in Cell Biology: Antibodies in Cell Biology, volume 37 (Asai, ed. 1993); Basic and Clinical Immunology (Stites & Ten, eds., 7th ed. 1991). Immunological binding assays (or immunoassays) typically use an antibody that specifically binds to a protein or antigen of choice (in this case the protein or antigenic subsequence thereof). As explained herein, the antibody may be produced by any of a number of means well known to those of skill in the art and as described above.

Immunoassays also often use a labeling agent to specifically bind to and label the complex formed by the antibody and antigen. The labeling agent may itself be one of the moieties comprising the antibody/antigen complex. Thus, the labeling agent may be a labeled protein or a labeled antibody. Alternatively, the labeling agent may be a third moiety, such a secondary antibody, that specifically binds to the antibody/protein complex (a secondary antibody is typically specific to antibodies of the species from which the first antibody is derived). Other proteins capable of specifically binding immunoglobulin constant regions, such as protein A or protein G may also be used as the label agent. These proteins exhibit a strong non-immunogenic reactivity with immunoglobulin constant regions from a variety of species (see, e.g., Kronval et al., J. Immunol. 111:1401-1406 (1973); Akerstrom et al., J. Immunol. 135:2589-2542 (1985)). The labeling agent can be modified with a detectable moiety, such as biotin, to which another molecule can specifically bind, such as streptavidin. A variety of detectable moieties are well known to those skilled in the art.

Throughout the assays, incubation and/or washing steps may be required after each combination of reagents. Incubation steps can vary from about 5 seconds to several hours, optionally from about 5 minutes to about 24 hours. However, the incubation time will depend upon the assay format, antigen, volume of solution, concentrations, and the like. Usually, the assays will be carried out at ambient temperature, although they can be conducted over a range of temperatures, such as 10° C. to 40° C.

Non-Competitive Assay Formats

Immunoassays for detecting protein in samples may be either competitive or noncompetitive. Noncompetitive immunoassays are assays in which the amount of antigen is directly measured. In one preferred “sandwich” assay, for example, the antibodies can be bound directly to a solid substrate on which they are immobilized. These immobilized antibodies then capture protein present in the test sample. Proteins thus immobilized are then bound by a labeling agent, such as a second antibody bearing a label. Alternatively, the second antibody may lack a label, but it may, in turn, be bound by a labeled third antibody specific to antibodies of the species from which the second antibody is derived. The second or third antibody is typically modified with a detectable moiety, such as biotin, to which another molecule specifically binds, e.g., streptavidin, to provide a detectable moiety.

Competitive Assay Formats

In competitive assays, the amount of protein present in the sample is measured indirectly by measuring the amount of a known, added (exogenous) protein displaced (competed away) from an antibody by the unknown protein present in a sample. In one competitive assay, a known amount of protein is added to a sample and the sample is then contacted with an antibody that specifically binds to protein. The amount of exogenous protein bound to the antibody is inversely proportional to the concentration of protein present in the sample. In a particularly preferred embodiment, the antibody is immobilized on a solid substrate. The amount of protein bound to the antibody may be determined either by measuring the amount of protein present in protein/antibody complex, or alternatively by measuring the amount of remaining uncomplexed protein. The amount of protein may be detected by providing a labeled molecule.

A hapten inhibition assay is another preferred competitive assay. In this assay the known protein is immobilized on a solid substrate. A known amount of antibody is added to the sample, and the sample is then contacted with the immobilized protein. The amount of antibody bound to the known immobilized protein is inversely proportional to the amount of protein present in the sample. Again, the amount of immobilized antibody may be detected by detecting either the immobilized fraction of antibody or the fraction of the antibody that remains in solution. Detection may be direct where the antibody is labeled or indirect by the subsequent addition of a labeled moiety that specifically binds to the antibody as described above.

Cross-Reactivity Determinations

Immunoassays in the competitive binding format can also be used for crossreactivity determinations. For example, a protein can be immobilized to a solid support. Proteins are added to the assay that compete for binding of the antisera to the immobilized antigen. The ability of the added proteins to compete for binding of the antisera to the immobilized protein is compared to the ability of the protein to compete with itself. The percent crossreactivity for the above proteins is calculated, using standard calculations. Those antisera with less than 10% crossreactivity with each of the added proteins listed above are selected and pooled. The cross-reacting antibodies are optionally removed from the pooled antisera by immunoabsorption with the added considered proteins, e.g., distantly related homologs.

The immunoabsorbed and pooled antisera are then used in a competitive binding immunoassay as described above to compare a second protein, thought to be perhaps an allele or polymorphic variant of a protein, to the immunogen protein. In order to make this comparison, the two proteins are each assayed at a wide range of concentrations and the amount of each protein required to inhibit 50% of the binding of the antisera to the immobilized protein is determined. If the amount of the second protein required to inhibit 50% of binding is less than 10 times the amount of the protein that is required to inhibit 50% of binding, then the second protein is said to specifically bind to the polyclonal antibodies generated to the immunogen.

Other Assay Formats

Western blot (immunoblot) analysis is used to detect and quantify the presence of protein in the sample. The technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind the protein. The antibodies specifically bind to the protein on the solid support. These antibodies may be directly labeled or alternatively may be subsequently detected using labeled antibodies (e.g., labeled sheep anti-mouse antibodies) that specifically bind to the antibodies.

Other assay formats include liposome immunoassays (LIA), which use liposomes designed to bind specific molecules (e.g., antibodies) and release encapsulated reagents or markers. The released chemicals are then detected according to standard techniques (see Monroe et al., Amer. Clin. Prod. Rev., 5:34-41 (1986)).

Reduction of Non-Specific Binding

One of skill in the art will appreciate that it is often desirable to minimize non-specific binding in immunoassays. Particularly, where the assay involves an antigen or antibody immobilized on a solid substrate it is desirable to minimize the amount of non-specific binding to the substrate. Means of reducing such non-specific binding are well known to those of skill in the art. Typically, this technique involves coating the substrate with a proteinaceous composition. In particular, protein compositions such as bovine serum albumin (BSA), nonfat powdered milk, and gelatin are widely used with powdered milk being most preferred.

Labels

The particular label or detectable group used in the assay is not a critical aspect of the invention, as long as it does not significantly interfere with the specific binding of the antibody used in the assay. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g., DYNABEADS™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic beads (e.g., polystyrene, polypropylene, latex, etc.).

The label may be coupled directly or indirectly to the desired component of the assay according to methods well known in the art. As indicated above, a wide variety of labels may be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to another molecules (e.g., streptavidin) molecule, which is either inherently detectable or covalently bound to a signal system, such as a detectable enzyme, a fluorescent compound, or a chemiluminescent compound. The ligands and their targets can be used in any suitable combination with antibodies that recognize the protein, or secondary antibodies.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidotases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that may be used, see U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple colorimetric labels may be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

### Pharmaceutical Compositions and Modes of Administration

Pharmaceutically acceptable carriers are determined in part by the particular compound, i.e., IGF-II inhibitor or blocking agent, being administered (e.g., nucleic acid, protein, small molecule, etc.) as well as by the particular method used to administer the compound. Accordingly, there are a wide variety of suitable formulations of pharmaceutical compositions of the present invention (see, e.g., Remington's Pharmaceutical Sciences, 17^{th} ed., 1989). Administration can be in any convenient manner, e.g., by injection, oral administration, inhalation, transdermal application, or rectal administration.

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The compound of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration and intravenous administration are the preferred methods of administration. The formulations of commends can be presented in unit-dose or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above.

The dose administered to a patient, in the context of the present invention, should be sufficient to effect a beneficial therapeutic response in the patient over time. The dose will be determined by the efficacy of the particular composition employed and the condition of the patient, as well as the body weight or surface area of the patient to be treated. The size of the dose also will be determined by the existence, nature, and extent of any adverse side-effects that accompany the administration of a particular compound, vector, or transduced cell type in a particular patient.

In determining the effective amount of the composition to be administered in the treatment (or prophylaxis) of tumorigenesis or angiogenesis, the physician evaluates circulating plasma levels of the composition, composition toxicities, progression of the disease, and the production of antibodies.

For administration, compounds, vectors and transduced cells of the present invention can be administered at a rate determined by the LD-50 of the IGF-II inhibitor, vector, or transduced cell type, and the side-effects of the inhibitor, vector or cell type at various concentrations, as applied to the mass and overall health of the patient. Administration can be accomplished via single or divided doses.

### Kits for Detection of IGF-II and EphB4

In another aspect, the present invention provides kits for qualifying tumor status, which kits are used to detect IGF-II, a IGF-II TK receptor (such as IGF-IR and/or InsR-A) and EphB4 according to the invention. In one embodiment, the kit comprises (i) a first solid support, such as a chip, a microtiter plate or a bead or resin, having a first capture reagent attached thereon, wherein the first capture reagent binds IGF-II, a biomarker of the invention; (ii) a second solid support, such as a chip, a microtiter plate or a bead or resin, having a second capture reagent attached thereon, wherein the second capture reagent binds a IGF-II TK receptor (such as IGF-IR and/or InsR-A), a biomarker of the invention; and (iii) a third solid support, such as a chip, a microtiter plate or a bead or resin, having a second capture reagent attached thereon, wherein the third capture reagent binds EphB4, a biomarker of the invention. Thus, for example, the kits of the present invention can comprise solid supports with an antibody that specifically binds IGF-II, an antibody that specifically binds a IGF-II TK receptor (such as IGF-IR and/or InsR-A) and an antibody that specifically binds EphB4. In another embodiment, the kits of the present invention can comprise mass spectrometry probes for SELDI, such as ProteinChip® arrays. In the case of biospecfic capture reagents, the kit can comprise a solid support with a reactive surface, and a container comprising the biospecific capture reagent.

The kit can also comprise a washing solution or instructions for making a washing solution, in which the combination of the capture reagent and the washing solution allows capture of IGF-II, the IGF-II TK receptor (e.g., IGF-IR and/or InsR-A) and EphB4 on the solid support for subsequent detection by, e.g., mass spectrometry. The kit may include more than one type of capture reagent (e.g., an adsorbent), each present on a different solid support.

In a further embodiment, such a kit can comprise instructions for suitable operational parameters in the form of a label or separate insert. For example, the instructions may inform a consumer about how to collect the sample, how to wash the solid support (e.g., probe) or the particular biomarkers (e.g., IGF-II, a IGF-II TK receptor and EphB4) to be detected.

In yet another embodiment, the kit can comprise one or more containers with IGF-II, a IGF-II TK receptor (such as IGF-IR and/or InsR-A) and EphB4 samples, to be used as standard(s) for calibration.

The invention will be described in greater detail by way of specific examples. The following examples are offered for illustrative purposes, and are not intended to limit the invention in any manner. Those of skill in the art will readily recognize a variety of non-critical parameters that can be changed or modified to yield essentially the same results.

### EXAMPLES

### Example 1

### Block of Secreted IGF-II by a Neutralizing Antibody Strongly Inhibits EphB4 Protein Expression in Mesothelioma Cell Lines Expressing Both the IGF-IR and the InsR Isoform A

Insulin-like growth factor-II has been found to be over-expressed and secreted in a systemically active form in a variety of solid tumors where it causes insulin-independent hypoglycemia (Schweichler et al., Obstet. Gynecol., 85(5):810-813 (1995); and Van Doom et al., Clin. Chem., 48(10):1739-1750 (2002)). Mesothelioma is described among these IGF-II secreting types of cancers (see, FIGS. 1C and 1D; and Schweichler et al., Obstet. Gynecol., 85(5):810-813 (1995)). The presence of the IGF-II transcript as well as one of the two Tyrosine kinase receptors known to mediate IGF-II cellular effects, namely, the IGF-IR and the InsR-A receptors, was confirmed in four different human mesothelioma cell lines (2052H, 211H, 2373H and 2028H) by RT-PCR (see, FIG. 1C). To further demonstrate the dependence of EphB4 protein expression on IGF-II in this type of cancer, mesothelioma cells were incubated for 24 hrs either in the presence or absence of an IGF-II neutralizing antibody, were harvested and then tested for expression of EphB4 by Western Blot. The results show a marked decrease in EphB4 expression, but not of a control protein (b-actin), demonstrating that in mesothelioma cells IGF-II is secreted in the extracellular medium and it self-induces and/or maintains the expression of EphB4 through IGF-IR and InsR-A activation.

### Example 2

### Venous Marker EphB4 is Regulated by IGF-II at the Transcriptional Gene Level and this Effect is Mediated by the TAAT Motif at Position −1365 of the Human EphB4 Promoter

In order to establish the role of IGF-II on EphB4 expression, a mouse embryo fibroblast cell line expressing the human IGF-I receptor (R⁺ cells) was transiently transfected with three variants of the human EphB4 promoter previously described by Bruhl et al., supra, along with trace amounts of a renilla luciferase mock vector. 24 hrs post-trasfection, the cells were serum starved for 12 hrs and then stimulated (or not) with 10 nM IGF-II for 8 hrs. Cell lysates were generated and then assayed for Luciferase activity using a Promega Dual luciferase assay and a Veritas Luminescence reader (Biotek).

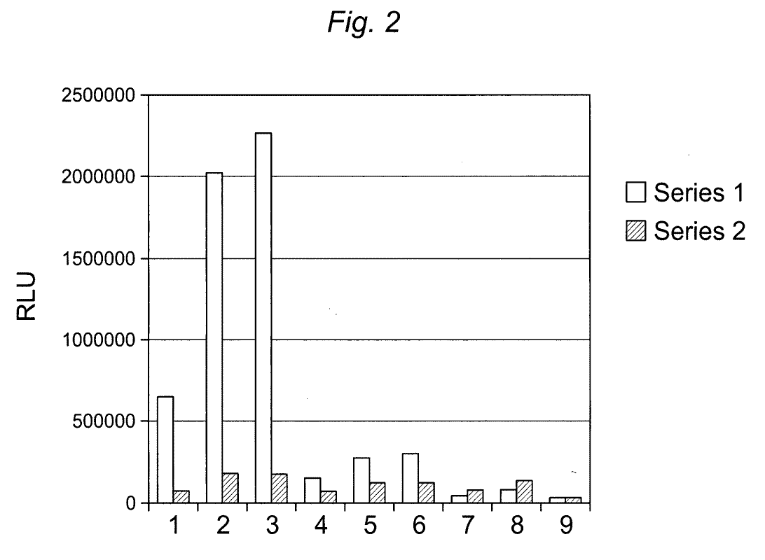

The results clearly demonstrate that IGF-II at low nanomolar concentrations is able to stimulate EphB4 gene activation at levels comparable to the ones obtained with serum activation (see, FIG. 2). This result is consistent with the previous finding of high IGF-1 levels in serum, which exert its cellular effect through the IGF-I receptors.

### Example 3

### IGF-II Induced Activation of EphB4 Gene Expression is Mediated by Activation of Either the IGF-IR or the InsR Isoform A at the Cellular Level

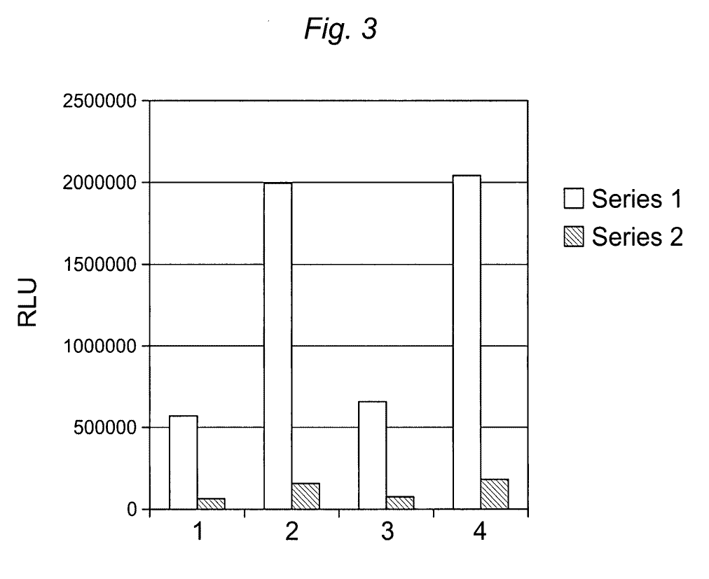

In order to confirm the presence of the above-described IGF-II-EphB4 activation loop through InsR activation, the same experiment described in Example 1 was performed in R-InsR-A cells, which are IGF-IR null embryo fibroblast cells expressing the human Insulin receptor short isoform (InsR-A) previously described (Frasca et al., Mol. Cell. Biol., 19(5):3278-3288 (1999)). As shown in FIG. 3, the result is comparable to that obtained through IGF-IR activation. Thus, this example further supports the role of IGF-II as a wider target than IGF-IR towards the block of EphB4-dependent effects in cancer.

### Example 4

### EphB4 is Ectopically Expressed in IGF-II Positive Cancer Cell Lines

In order to establish a link between IGF-II and EphB4 during malignant transformation, a number of cancer cells that have already tested positive for IGF-II were also screened for EphB4 expression through RT-PCR.

As shown in FIG. 4, EphB4 is strongly expressed in overtly malignant cancer cell lines already known to express either IGF-IR or InsR-A. This finding establishes a novel role for the combined detection of IGF-II, a IGF-II TK receptor (IGF-IR and/or InsR-A) and EphB4 in defining tumor staging were the presence of both factors correlates with an advanced stage of cancer, whereas the lack of IGF-II expression correlates with an early stage of cancer and/or with a non-cancerous condition. The presence of IGF-II in surrounding stromal tissue in the presence of EphB4 expression in the tumor correlates with a pre-malignant stage.

It is to be understood that the above description is intended to be illustrative and not restrictive. Many embodiments will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated herein by reference for all purposes.

What is claimed is:

1. A method for qualifying the tumor status in a subject, said method comprising:
(a) detecting the presence or absence of IGF-II, a IGF-II Tyrosine Kinase receptor (“IGF-II TK receptor”) and EphB4 in a biological sample from the subject; and
(b) correlating the presence or absence of IGF-II, the IGF-II TK receptor and EphB4 with tumor status.

2. The method in accordance with claim 1, wherein the IGF-II TK receptor is a member selected from the group consisting of IGF-IR, InsR-A or both IGF-IR and InsR-A.

3. The method in accordance with claim 1, wherein the tumor status is benign and the biological sample is IGF-II negative/IGF-II TK receptor positive/EphB4 negative.

4. The method in accordance with claim 1, wherein the tumor status is transitional and the biological sample is IGF-II negative/IGF-II TK receptor positive/EphB4 positive.

5. The method in accordance with claim 4, further comprising detecting the presence or absence of IGF-II in stromal or parenchymal tissue surrounding the tumor, wherein the presence of IGF-II in surrounding stromal or parenchymal tissue further indicates that the tumor status is transitional.

6. The method in accordance with claim 1, wherein the tumor status is malignant and the biological sample is IGF-II positive/IGF-II TK receptor positive/EphB4 positive.

7. The method in accordance with claim 1, wherein IGF-II, the IGF-II TK receptor and EphB4 are detected by detecting RNA.

8. The method in accordance with claim 1, wherein IGF-II, the IGF-II TK receptor and EphB4 are detected by detecting expressed protein.

9. The method in accordance with claim 1, wherein IGF-II, the IGF-II TK receptor and EphB4 are detected by immunoassay.

10. The method in accordance with claim 1, wherein the biological sample is a tumor cell.

11. The method in accordance with claim 10, wherein the tumor cell is selected from the group consisting of a carcinoma of the breast, kidney, thyroid, prostrate, liver, bone, lymph node, cervix, colon and skin or a sarcoma of the bone, muscle and lymphatic tissue.

12. The method in accordance with claim 1, wherein the biological sample is a biopsy of a solid tumor.

13. The method in accordance with claim 1, further comprising (c) managing subject treatment based on the tumor status.

14. The method in accordance with claim 1, further comprising (c) reporting the tumor status to the subject.

15. The method in accordance with claim 1, further comprising (c) recording the tumor status on a tangible medium.

16. The method in accordance with claim 13, further comprising (d) detecting IGF-II, the IGF-II TK receptor and EphB4 after subject management and correlating the measurement with disease progression.

17. A method for identifying a compound that modulates angiogenesis in a cell that co-expresses IGF-II, a IGF-II TK receptor and EphB4, said method comprising the steps of:
(i) contacting said tumor cell with a compound that inhibits IGF-II; and
(ii) determining the functional effect of the compound upon EphB4.

18. The method in accordance with claim 17, wherein the compound inhibits IGF-II gene expression.

19. The method in accordance with claim 17, wherein the compound inhibits IGF-II protein expression.

20. The method in accordance with claim 17, wherein the compound is an antibody.

21. The method in accordance with claim 17, wherein the compound is an RNAi molecule.

22. The method in accordance with claim 17, wherein the compound is an antisense molecule.

23. The method in accordance with claim 17, wherein the compound is a small organic molecule.

24. The method in accordance with claim 17, wherein modulation is inhibition of angiogenesis.

25. The method in accordance with claim 17, wherein the functional effect is EphB4 protein expression.

26. A method for identifying a compound that inhibits tumorigenesis in a tumor cell that co-expresses IGF-II, a IGF-II TK receptor and EphB4, said method comprising the steps of:
(i) contacting said tumor cell with a compound that inhibits IGF-II; and
(ii) determining the functional effect of the compound upon EphB4.

27. The method in accordance with claim 26, wherein the compound inhibits IGF-II gene expression.

28. The method in accordance with claim 26, wherein the compound inhibits IGF-II protein expression.

29. The method in accordance with claim 26, wherein the compound is an antibody.

30. The method in accordance with claim 26, wherein the compound is an RNAi molecule.

31. The method in accordance with claim 26, wherein the compound is an antisense molecule.

32. The method in accordance with claim 26, wherein the compound is a small organic molecule.

33. The method in accordance with claim 26, wherein the functional effect is EphB4 gene expression.

34. The method in accordance with claim 26, wherein the functional effect is EphB4 protein expression.

35. A kit for qualifying tumor status in a subject, the kit comprising:
(a) a first solid support comprising at least one capture reagent attached thereto, wherein the capture reagent binds IGF-II;
(b) a second solid support comprising at least one capture reagent attached thereto, wherein the capture reagent binds IGF-II TK receptor;
(c) a third solid support comprising at least one capture reagent attached thereto, wherein the capture reagent binds EphB4; and
(d) instructions for using the first and second solid supports to detect IGF-II, the IGF-II TK receptor and EphB4.

36. The kit in accordance with claim 35, wherein the capture reagent that binds IGF-II is an antibody.

37. The kit in accordance with claim 35, wherein the capture reagent that binds the IGF-II TK receptor is an antibody.

38. The kit in accordance with claim 35, wherein the IGF-II TK receptor is a member selected from the group consisting of IGF-IR, InsR-A and a combination of IGF-IR and InsR-A.

39. The kit in accordance with claim 35, wherein the capture reagent that binds EphB4 is an antibody.

40. The kit in accordance with claim 35, wherein the capture reagent that binds EphB4 is EphrinB2.

41. The kit in accordance with claim 35, further comprising (d) a container containing IGF-II, the IGF-II TK receptor or EphB4.

42. A method for inhibiting EphB4 gene or protein expression in a cell that co-expresses EphB4, a IGF-II TK receptor and IGF-II, said method comprises contacting said cell with a compound that inhibits IGF-II, thereby inhibiting EphB4 gene or protein expression.

43. The method in accordance with claim 42, wherein the compound inhibits IGF-II gene expression.

44. The method in accordance with claim 42, wherein the compound inhibits IGF-II protein expression.

45. The method in accordance with claim 42, wherein the cell is a tumor cell.

46. A method for inhibiting the growth of a tumor cell that co-expresses EphB4, a IGF-II TK receptor and IGF-II, said method comprises contacting said tumor cell with a compound that inhibits IGF-II, thereby inhibiting the growth of said tumor cell.

47. The method in accordance with claim 46, wherein the compound inhibits IGF-II gene expression.

48. The method in accordance with claim 46, wherein the compound inhibits IGF-II protein expression.

49. The method in accordance with claim 46, wherein the compound is a member selected from the group consisting of an antibody, an antisense molecule, and RNAi molecule and a small organic molecule.

50. The method in accordance with claim 46, wherein said tumor cell is in a human.

51. An EphB4-specific targeting composition, said composition comprising an EphB4 binding moiety and an IGF-II inhibitor, wherein the EphB4 binding moiety is linked to the IGF-II inhibitor.

52. The EphB4-specific targeting composition in accordance with claim 51, wherein the EphB4 binding moiety is an antibody.

53. The EphB4-specific targeting composition in accordance with claim 51, wherein the EphB4 binding moiety is EphrinB2.

54. The EphB4-specific targeting composition in accordance with claim 51, wherein the IGF-II inhibitor is a member selected from the group consisting of an antibody, a peptide, an antisense molecule, a RNAi molecule and a small organic molecule

55. The EphB4-specific targeting composition in accordance with claim 51, wherein the IGF-II inhibitor is an antibody.

56. The EphB4-specific targeting composition in accordance with claim 51, wherein the EphB4 binding moiety is EphrinB2 and the IGF-II inhibitor is an antibody.

57. A method of inhibiting tumorigenesis in a cell that co-expresses IGF-II, a IGF-II TK receptor and EphB4, said method comprising contacting said cell with an EphB4-specific targeting compound, said compound comprising: (i) an EphB4 binding moiety; and (ii) an IGF-II inhibitor, wherein said EphB4 is linked to said IGF-II inhibitor.

58. An EphB4-specific targeting vehicle, said vehicle comprising:
a liposome;
a EphB4 binding moiety, wherein said EphB4 binding moiety is on the outer surface of said liposome; and
an IGF-II inhibitor.

59. The EphB4-specific targeting vehicle in accordance with claim 58, wherein the EphB4 binding moiety is an antibody.

60. The EphB4-specific targeting vehicle in accordance with claim 58, wherein the EphB4 binding moiety is EphrinB2.

61. The EphB4-specific targeting vehicle in accordance with claim 58, wherein the IGF-II inhibitor is a member selected from the group consisting of an antibody, a peptide, an antisense molecule, a RNAi molecule and a small organic molecule.

62. The EphB4-specific targeting vehicle in accordance with claim 58, wherein the IGF-II inhibitor is an antibody.

63. The EphB4-specific targeting vehicle in accordance with claim 58, wherein the EphB4 binding moiety is EphrinB2 and the IGF-II inhibitor is an antibody.

64. A method of inhibiting tumorigenesis in a cell that co-expresses IGF-II, a IGF-II TK receptor and EphB4, said method comprising contacting said cell with an EphB4-specific targeting vehicle, said vehicle comprising: (i) a liposome; (ii) a EphB4 binding moiety, wherein said EphB4 binding moiety is on the outer surface of said liposome; and (iii) an IGF-II inhibitor.

65. A method for inhibiting the progression of a tumor cell towards a malignant state, wherein EphB4 expression is driven by IGF-II expression in surrounding parenchymal or stromal tissue, said method comprises contacting said surrounding parenchymal or stromal tissue with a compound that inhibits IGF-II, thereby inhibiting the progression of said tumor cell towards a malignant state.
